SEQUENCE LISTING (27 sequences)

SEQ ID NO: 1 - protein, 25 aa, synthetic construct
ASRNKANDYTTEYSASVKGRFIVSR

SEQ ID NO: 2 - protein, 25 aa, synthetic construct
RIRNQAYSYTTEYAASVKGRFTISR

SEQ ID NO: 3 - protein, 14 aa, synthetic construct
VAYISSGGSSINYA

SEQ ID NO: 4 - protein, 25 aa, synthetic construct
RSVIFRGKVSASYETTYDNAKNRSA

SEQ ID NO: 5 - protein, 52 aa, synthetic construct
RSVIFRGKVSASYETTYDNAKNRSAGGRSVIFRGKVSASYETTYDNAKNRSA

SEQ ID NO: 6 - protein, 14 aa, synthetic construct
AYNISSGGSSIYAY

SEQ ID NO: 7 - protein, 30 aa, synthetic construct
AYNISSGGSSIYAYGGAYNISSGGSSIYAY

SEQ ID NO: 8 - protein, 25 aa, synthetic construct
ASRKKANDYTTEYSASVKGRFIVSR

SEQ ID NO: 9 - protein, 25 aa, synthetic construct
ASRNKARDYTTEYSASVKGRFIVSR

SEQ ID NO: 10 - protein, 25 aa, synthetic construct
ASRNKANDYTTEYSASVHGRFIVSR

SEQ ID NO: 11 - protein, 52 aa, synthetic construct
ASRKKANDYTTEYSASVKGRFIVSRGGASRKKANDYTTEYSASVKGRFIVSR

SEQ ID NO: 12 - protein, 52 aa, synthetic construct
ASRNKARDYTTEYSASVKGRFIVSRGGASRNKARDYTTEYSASVKGRFIVSR

SEQ ID NO: 13 - protein, 52 aa, synthetic construct
ASRNKANDYTTEYSASVHGRFIVSRGGASRNKANDYTTEYSASVHGRFIVSR

SEQ ID NO: 14 - protein, 52 aa, synthetic construct
ASRNKANDYTTEYSASVKGRFIVSRGGASRNKANDYTTEYSASVKGRFIVSR

SEQ ID NO: 15 - protein, 30 aa, synthetic construct
VAYISSGGSSINYAGGVAYISSGGSSINYA

SEQ ID NO: 16 - protein, 17 aa, synthetic construct
GAAVAYISSGGSSINYA

SEQ ID NO: 17 - protein, 35 aa, synthetic construct
GAAVAYISSGGSSINYAGGAAVAYISSGGSSINYA

SEQ ID NO: 18 - protein, 17 aa, synthetic construct
AYNISSGGSSIYAVAAG

SEQ ID NO: 19 - protein, 35 aa, synthetic construct
AYNISSGGSSIYAVAAGGAYNISSGGSSIYAVAAG

SEQ ID NO: 20 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 451 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 214 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0336696 A1
(43) Pub. Date: Oct. 10, 2024

(54) HOMOLOGOUS DIMERIZATION PEPTIDES AND ANTIBODIES COMPRISING THE SAME

(71) Applicant: PXRADIA MAB TECHNOLOGIES INC., Vancouver (CA)

(72) Inventor: Alton C. MORGAN, Vancouver (CA)

(21) Appl. No.: 18/579,715

(22) PCT Filed: Jul. 13, 2022
(86) PCT No.: PCT/CA2022/051093

§ 371 Date: Jan. 16, 2024

Related U.S. Application Data
(60) Provisional application No. 63/221,686, filed on Jul. 14, 2021.

(51) Int. Cl. C07K 16/28; A61K 39/00; A61P 35/00; C07K 14/47; C07K 16/22; C07K 16/32; G01N 33/574

(52) U.S. Cl. CPC C07K 16/2887; A61P 35/00; C07K 14/47; C07K 16/22; C07K 16/2827; C07K 16/32; G01N 33/57434; A61K 2039/505; C07K 2317/24; C07K 2317/31; C07K 2317/35; C07K 2317/565; C07K 2317/732; C07K 2317/734

(57) ABSTRACT

Homologous dimerization (HD) peptides are described having improved self-binding. The HD peptides may comprise an amino acid sequence having the reverse configuration compared to a corresponding naturally occurring HD peptide or comprise the amino acid sequence of a naturally occurring HD peptide with one or more hydrophilic substitutions. Furthermore dimers of HD peptides are also described. Antibodies comprising said HD peptides or HD dimers are also disclosed, as well as methods of manufacture and uses of said antibodies.

### FIELD OF INVENTION

The present invention relates to homologous dimerization peptides, antibodies comprising said homologous dimerization peptides, and methods and uses thereof.

### BACKGROUND OF THE INVENTION

Antibodies have been praised as “magic bullets” to combat disease and have emerged as a major therapeutic tool for the treatment of chronic diseases, such as cancer and autoimmune disorders. Notable success stories include Herceptin® in the treatment of breast cancer and Rituxan® in the treatment of non-Hodgkin's lymphoma. A key advantage of antibodies in the treatment of disease lies in their ability to target disease-causing cells or molecules, while sparing healthy tissues and normal products of the body.

Currently there are sixteen FDA approved monoclonal antibodies accounting for $62 Bn in annual sales. This is expected to grow to 70 approved products generating $120 Bn in annual sales by 2025. Moreover, the newest and most successful cancer therapy of the last decade, monoclonal antibodies (mAbs) directed at suppressor molecules on the surface of cancer cells such as PD-L1 have proven to be able to release the brakes on a cancer patient's immune system resulting in high therapeutic activity in cancers previously not well treated by traditional chemotherapy. This target alone is expected to generate annual sales of $35 Bn by 2025.

However, antibodies that exhibit desired specificities in laboratory studies often fail in pre-clinical and clinical evaluations because of inefficient targeting, low biological activity, low therapeutic efficacy, and/or unacceptable side effects. This is in part due the fact that antibodies represent only one arm of the immune defense, where T-cells provide the other strategy in immune defense.

Antibodies are ideal platforms for targeting and delivery devices. Antibodies have been used as delivery devices for several biologically active molecules, such as toxins, drugs and cytokines (ADC). In some cases fragments of antibodies, antigen binding fragments (Fab) or single chain variable fragments (scFv), are preferred because of better tissue penetration. Despite the fact that there are now several approved ADC, their development has been very costly requiring long approval times.

A preferred therapeutic mAb form is a human (or humanized) IgG1 wherein, the mAbs therapeutic activity is imparted by its binding (agonist, antagonistic,) or effector functions [Complement-mediated cytotoxicity (C′MC), Antibody-dependent cell-mediated toxicity (ADCC) or triggering of apoptosis]. mAb are already adapted for long survival in blood, have sites which help vascular and tissue penetration and are functionally linked with a number of defense mechanisms of the innate immunity.

It is known that a major mechanism by which therapeutic antibodies are effective against their target cells is by inducing cell death, i.e., antibody-induced apoptosis. Such induced apoptosis is typically triggered by crosslinking receptors that are part of the cell's apoptosis signal pathway. For example, crosslinking the B-cell antigen receptor by means of antibodies induces apoptosis in B-cell tumors (Ghetie M., et al., 1997). Crosslinking of cellular receptors also increases the binding avidity of an antibody to its target antigen, and thus is likely to increase all cell surface-dependent therapeutic mechanisms, such as complement-mediated killing and complement-dependent opsonization and phagocytosis, antibody-dependent cellular cytotoxicity (ADCC), as well as enhanced inhibition of cell growth or alterations in metabolic pathways within cells through increased binding to and blockade of cellular receptors when using antibodies targeted to cellular receptors.

The therapeutic properties of the antibodies can be enhanced with respect to affinity for its target antigen by using Fab libraries aimed at “evolving” the native antibody. This can result in the increase of affinity by sometimes as much as 100 to 1000 fold. However, this does not overcome the basic nature of the binding of monoclonal antibodies. With regard to protein epitopes, monoclonal antibodies typically bind with one arm to a single epitope—when a mAb disassociates from its target the next target is typically too far away for the mAb to rebind. Such a handicap can be overcome by using a target system in which the mAb can span the distance between epitopes: this requires typically very high antigen density clustered on the membrane. An example is cell surface immunoglobulin on B-cells. Unfortunately there are very few therapeutic targets of this nature.

Antigen binding can be enhanced by increasing the opportunity for a mAb to cross-link its target, engaging epitopes simultaneously on neighboring target antigens. With many targets this cross-linking is a potent means to trigger apoptosis. The potential for cross-linking can be enhanced by increasing valency of antibodies such as with pentameric IgM antibodies. This can also be done through recombinant means creating multimeric immunoglobulin molecules from IgG (Xiao-Yun Liu, Laurentiu M. Pop, Lydia Tsai, Iliodora V. Pop and Ellen S. Vitetta, Int. J. Cancer 129, 497-506 (2011)). However with most therapeutic targets, cross-linking cannot be achieved even with an increase in valency and size of the immunoglobulin molecule.

Valency and avidity is increased in a rare class of self-binding or homophilic antibodies, variously known as “autophilic antibodies” or “autobodies”, which have been identified in Nature (Kang, C. Y., Cheng, H. L., Rudikoff, S. and Kohler, H. J. Exp. Med. 165:1332, (1987): Xiyun, A. N., Evans, S. V., Kaminki, M. J., Fillies, S. F. D., Reisfeld, R. A., Noughton, A. N. and Chapman, P. B. J. Immunol. 157:1582-1588 (1996)). This originates as a result of secondary (to antigen binding) interactions and can incorporate multiple IgG and span any distance on the cell surface between targets. They are capable of forming dimers and/or polymers through noncovalent interactions with self. One example of an autophilic antibody is TEPC-15 (T15), which targets a normally cryptic determinant of phosphorylcholine on apoptotic cells and atherosclerotic lesions (Binder, J., et al., 2003: Kang. C-Y, et al., 1988). Dimerization or multimerization may be induced only after the modified antibody attaches to its cell surface target, i.e., “differential oligomerization”. In solution, an autophilic antibody can be in equilibrium between its monomeric and dimeric forms (Kaveri S., et al., 1990). Unfortunately, Nature has created very few of these types of antibodies and they are to a limited number of targets.

A peptide in the heavy chain region of the TEPC-15 (T15) antibody was identified as self-binding and imparting higher therapeutic activity to the antibody (Kang. C. Y. Brunck, T. K., Kieber-Emmons, T., Blalock, J. E. and Kohler, H., Science, 240:1034-1036, 1988). Such peptides are known as “autophilic peptides” or “homologous dimerization (HD) peptides”. Elucidation of this peptide sequence and others with similar ability to induce self-association of antibodies offered the opportunity for imparting the same property of self-association to other antibodies targeting different antigens. More recently the nature of self-binding was explored and the preference for the synthetic form of the peptide to form secondary and tertiary features was deduced (Bost K L, Blalock J E. Viral Immunol. 2 (4), 229-238 (1989); Kohler, H, Immunotherapy (2013) 5 (3), 235-246).

In efforts to enhance antigen detection and/or therapeutic efficacy of known antibodies, hybrid molecules comprising two distinct covalently linked domains have been proposed. For example, U.S. Patent Pub. No. 2003/0103984 (Kohler) and U.S. Patent Pub. No. 2004/0185039 (Kohler) disclose a fusion proteins comprising antibody and peptide domains in which the peptide domain can have autophilic activity. WO 2009/002939 discloses an immunoglobulin component having binding affinity for a CD-20 antigen fused to an autophilic peptide. WO 2009/108803 disclose methods and kits for detecting analytes in a sample using an antibody conjugated to an autophilic peptide.

However, there is still a need for improving sensitivity and efficacy of antibodies for detection, prevention and/or treatment of disease.

### SUMMARY OF THE INVENTION

The present invention relates to homologous dimerization (HD) peptides having improved biological activity of self-association or homologous dimerization. The invention also relates to fusion proteins (e.g. chemically conjugated or recombinant antibodies) comprising said HD peptides. The fusion proteins comprising an immunoglobulin component or antibody and the HD peptide may be recombinant or the HD peptide may be conjugated thereto, in a manner that does not disrupt antigen binding and that allows preferred conformation changes in the HD peptide sequence thereby imparting dimerization activity. The HD peptides disclosed herein may be used to enhance the potency of therapeutic antibodies or to increase binding sensitivity and/or avidity for other application such as diagnostics.

In one aspect it is provided a homologous dimerization (HD) peptides comprising an amino acid sequence in reverse configuration compared to a corresponding naturally occurring HD peptide, or conservative variants thereof. In some embodiments, the corresponding naturally occurring HD peptide may be a T15 HD peptide or a R24 HD peptide.

In some embodiments, the HD peptide may comprise an amino acid sequence having at least 90% sequence identity to RSVIFRGKVSASYETTYDNAKNRSA (SEQ ID No: 4) or AYNISSGGSSIYAY (SEQ ID No. 6), i.e. the reverse amino acid sequence of T15 and R24 HD peptides.

It a further aspect it is provide a homologous dimerization (HD) peptides comprising one or more than one substitutions imparting improved self-binding properties, or conservative variants thereof. The one or more than one substitutions may be hydrophilic substitutions. Accordingly the HD peptide as described herewith may comprise one or more than one amino acid substitutions wherein the one or more than one substitution increases the hydropathy of the HD peptide.

In some embodiments, the homologous dimerization (HD) peptide may comprise the amino acid sequence ASRNKANDYTTEYSASVKGRFIVSR (SEQ ID No: 1), having one or more substitutions at nucleotide positions 4, 7, and 18. The one or more than one amino acid substitutions at position 4 may be to K or a conserved substitution of K, the one or more than one amino acid substitutions at position 7 may be to R or a conserved substitution of R and the one or more than one amino acid substitutions at position 18 may be to H or a conserved substitution of H.

In some embodiments, the one or more than one substitutions may be N4K, N7R and/or K18H.

In some embodiments, the HD peptide may comprise an amino acid sequence having at least 90% sequence identity to SEQ ID NO: 8, 9 or 10.

In yet another aspect it is provide homologous dimerization (HD) peptide dimer comprising HD peptides as described above. The HD peptides of the dimer may be joined by a linker. In some embodiments, the linker may be gly-gly.

It is further provided a homologous dimerization (HD) peptide dimer comprising a first HD peptide and a second HD peptide, wherein the first and second HD peptide are derived from a naturally occurring HD peptide or wherein the first and second HD peptide are derived from a reverse sequences of a naturally occurring HD peptide.

In some embodiments, the HD peptide dimer may comprise an amino acid sequence having at least 90% sequence identity to SEQ ID No. 5 or 7, i.e. a dimer of T15 or R24 HD peptides joined by a gly-gly linker.

In another embodiments, the HD peptide dimer may comprise an amino acid sequence having at least 90% sequence identity to SEQ ID No. 11, 12, 13, 14 or 15. In one embodiment the HD peptide dimer comprises an amino acid sequence having 80-100% sequence identity to SEQ ID NO: 5, 7, 11, 12, 13, 14, 15, 17 or 19.

The invention further provides an antibody or antigen-binding fragment comprising the HD peptide or HD peptide dimers described herein fused thereto. Antibodies with the HD peptides or HD peptide dimer described herein have been found to impart improved binding and therapeutic properties.

In some embodiments, the antibody or antigen-binding fragment is a humanized IgG. For example, the antibody or antigen-binding fragment may be humanized IgG1, humanized IgG4 or humanized IgG3.

The HD peptide or HD peptide dimer may be located at an appropriate site in the antibody. In some embodiments, the HD peptide or HD peptide dimer may be fused to a nucleotide affinity site of the antibody or antigen-binding fragment. For example, the HD peptide or HD peptide dimer may be fused through lysines, cysteines or carbohydrates.

In some embodiments, the HD peptide or HD peptide dimer is positioned immediately following the CDR3 of the heavy chain or light chain of the antibody. In alternative embodiments, the HD peptide or HD peptide dimer is positioned immediately following the C-terminal of a heavy chain or light chain constant region of the antibody. In alternative embodiments, the HD peptide or HD peptide dimer is positioned immediately following the heavy chain variable region of the antibody. In alternative embodiments, the HD peptide or HD peptide dimer is positioned immediately to a C-terminal of a Fc region of the antibody.

The antibodies described herein may be created by conjugating an HD peptide or HD peptide dimer thereto. Alternatively, the antibodies may be created by recombinant methods. The present invention also relates to methods for creating recombinant antibody and a peptide having the ability to self associate forming lattices and cross-linking their target antigen. Such cross-linking can then lead to enhanced cell signaling, enhanced receptor blockade, internalization of antigen/antibody complexes and even induction of apoptosis.

In some embodiments, the attachment position of the HD peptide or HD peptide dimer to the antibody may be preceded by a spacer, such as gly-gly.

The antibody or antigen-binding fragment (Fab) may be a single-chain antibody (scFvs), bi-specific antibody (BsAbs) or antibody-like peptide.

In some embodiments, the antibody is a humanized monoclonal antibody. The antibody may be a Her-2neu antibody, such as Herceptin. The antibody may be a CD-20 antibody, such as Rituxin. The antibody may be a vascular endothelial growth factor antibody, such as Avastin. The antibody may be a checkpoint inhibitor antibody, such as PD-L1.

There is further provided a composition comprising one or more antibody or antigen-binding fragment described herein, and a pharmaceutically acceptable carrier.

There is further provided an expression vector comprising a first nucleic acid sequence encoding the HD peptide or HD peptide dimer described herein. In some embodiments, the expression vector further comprises a second nucleic acid sequence encoding an antibody or antigen binding fragment, such that when the first and second nucleic acid sequences are expressed the HD peptide or HD peptide dimer and the antibody or antigen binding fragment are expressed as a fusion protein.

There is provided a method of generating a homologous dimerization (HD) antibody, comprising expressing the expression vector described herein in a host cell. For example, the host cell may be an animal cell, a yeast cell or a plant cell.

There is provided an isolated host cell transformed with the expression vector described herein.

There is provided a method of enhancing binding and/or potency of an antibody, comprising: conjugating a HD peptide or HD peptide dimer described herein to the antibody: or recombinantly expressing the antibody with the HD peptide or HD peptide dimer.

There is provided a method of treating a patient suffering from a disease or condition, comprising administering an antibody or antigen binding fragment described herein. The disease or condition may be one selected from the list consisting of cancer, auto-immune disorders, inflammatory disorders, neurodegenerative disease, cardiovascular disease, graft or transplant rejection.

There is provided a method of detecting an analyte in a sample, comprising: contacting the analyte with an antibody or antigen binding fragment directed to the analyte, wherein the antibody or antigen binding fragment is fused to the HD peptide or HD peptide dimer described herein: and detecting a complex formed by the analyte and the antibody fused to the HD peptide or HD peptide dimer.

There is provided a kit for detecting an analyte in a sample, comprising: an antibody or antigen binding fragment directed to the analyte, the antibody or antigen binding fragment fused to the HD peptide or HD peptide dimer described herein; and instructions for use in detecting the analyte.

There is provided a phage display library comprising an antibody or antigen binding fragment linked to a HD peptide or HD peptide dimer described herein.

The antibodies described herein may be used in therapy, for example, the prophylaxis and/or treatment of disease. The antibodies described herein may also be used in diagnostics, for example, in vitro diagnostic assays.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows (from left to right): (A) the amino acid sequence of the homophilic domain in T15 antibody (aa 50-70), indicating the CDR2 and Framework 3; (B) carbon background tracing of the T15 sequence aa 50-70 in the fold of the MPC 603 Fab structure: (C) ball representation of the T15 aa 50-70 MPC603 structure: (D) alignment of the T15 peptide showing hyrdopathic interactions;

FIG. 2 shows the hydropathic profile of the T15 HD peptide (T15) and its reversed sequence (rs-T15) using the Kyte-Doolittle algorithm with a window of 7 residues;

FIG. 3 shows the hydropathic alignments of the T15 peptide and its reversed sequence (rs-T15) as profiled in FIG. 2;

FIG. 4 shows the hydropathic analysis, amino acid by amino acid of the T15 peptide in 3 germline sequences drawn from the heavy chain CDR2/framework3 of Mopc antibodies with no, little or high HD activity. Top peptide: T15 peptide with amino acids contributing to self-binding illustrated in grey-scale. Hydropathic score of individual amino acid shown above or below. Middle peptide: Low HD binding, amino acid substitutions from T15 illustrated in grey-scale. Bottom peptide: No HD binding (Mpc 167), amino acid substitutions from T15 illustrated in grey-scale:

FIG. 5 shows the temperature dependent self-association for G11, S107 and G9 antibodies at 4° C. and 37° C. under non-physiologic conditions (see Bryan, J. A. and Kohler, H. Physical and Biological Properties of Homophilic therapeutic Antibodies, Cancer Immunology Immunotherapy, 60:507, 2010). The time required for meniscus to reach equilibrium after top-down to horizontal movement was measured. Error bars represent percent variations of two runs:

FIG. 6 shows human lymphoma cells with binding of Rituxin and HD-Rituxin detected by flow cytometry. Rituxin identifies 2 primary populations of cancer cells, one with a low antigen density (second arrow) and a second with higher density (third arrow):

FIG. 7 shows the complement dependent cytotoxicity of Rituxin and HD-Rituxin. Rituxin can mediate C′MC against 3 different cell lines (Raji, Ramon and JOK1) with different antigen density (light bar). C′MC by HD-Rituxin is significantly enhanced demonstrating higher levels of killing at lower antibody concentrations (dark bar). Antibody-dependent Cell-Mediated Cellular Cytotoxicity (ADCC) is also enhanced with HD-antibodies (similar results were obtained with HD-Herceptine, data not shown).

FIG. 8 shows the efficacy of Herceptin and HD-Herceptin in a nude mouse model of a low antigen expressing breast cancer (MCF-7), 7 days after injection of tumor;

FIG. 9 shows an HD peptide sequence from antibody R24. The interaction of the amino acids within the hairpin loop resulting in self binding is also illustrated:

FIG. 10 shows the hydropathic profile of the R24 HD peptide (R24) using the Kyte-Doolittle algorithm:

FIG. 11 shows detection of PSA antibodies using electro-chemiluminescence (e.g. for diagnostics). The graphs shows the signal generated with an anti-PSA antibody, modified by HD technology versus unmodified antibody.

### DETAILED DESCRIPTION

The following description is of a preferred embodiment.

The present disclosure relates to homologous dimerization (HD) peptides. More specifically, the present disclosure relates to artificial or synthetic homologous dimerization (HD) peptide. The HD peptides of the present disclosure have improved biological activity of self-association or homologous dimerization.

In one aspect the HD peptide may be an artificial or synthetic HD peptide that is derived from a naturally occurring HD peptide or a conservative variants thereof. For example the HD peptide may comprise an amino acid sequence in reverse configuration compared to a corresponding naturally occurring HD peptide, or a conservative variants thereof. In one embodiment the artificial or synthetic HD peptide may comprise an amino acid sequence that is reverse to the sequence of a T15 HD peptide or a R24 HD peptide.

It a further aspect it is provide a homologous dimerization (HD) peptides comprising one or more than one substitutions imparting improved self-binding properties, or conservative variants thereof. The one or more than one substitutions may be hydrophilic substitutions. The homologous dimerization (HD) peptide may comprise one or more than one amino acid substitutions wherein the one or more than one substitution increases the hydropathy of the HD peptide.

In some embodiments, the homologous dimerization (HD) peptide may comprise the amino acid sequence ASRNKANDYTTEYSASVKGRFIVSR (SEQ ID No: 1), having one or more substitutions at nucleotide positions 4, 7, and 18. The one or more than one amino acid substitutions at position 4 may be to K or a conserved substitution of K, the one or more than one amino acid substitutions at position 7 may be to R or a conserved substitution of R and the one or more than one amino acid substitutions at position 18 may be to H or a conserved substitution of H. The HD peptide may comprise an amino acid sequence having 80%-100% sequence identity to SEQ ID NO: 8, 9 or 10.

It is also provided artificial or synthetic HD peptide dimer that may comprise dimer of a naturally occurring HD peptide, dimer of a reverse sequence of a naturally occurring HD peptide or a conservative variants thereof. The homologous dimerization (HD) peptide dimer may comprise a first HD peptide and a second HD peptide, wherein the first and second HD peptide are derived from a naturally occurring HD peptide or wherein the first and second HD peptide are derived from a reverse sequences of a naturally occurring HD peptide. The first and second HD peptides of the dimer may be joined by a linker. The HD peptides in the dimer may further comprise one or more than one substitution as described herewith. The homologous dimerization (HD) peptide dimer may comprises an amino acid sequence having 80-100% sequence identity to SEQ ID NO: 5, 7, 11, 12, 13, 14, 15, 17 or 19.

Accordingly, the artificial or synthetic HD dimer and may comprise a first HD peptide and a second HD peptide, wherein the first, the second or the first and second HD peptide are derived from a naturally occurring HD peptide or a conservative variants thereof. Furthermore, the artificial or synthetic HD may be an HD dimer and comprise a first HD peptide and a second HD peptide, wherein the first, the second or the first and second HD peptide are derived from a reverse sequence of a naturally occurring HD peptide, or a conservative variants thereof. For example, the first and second HD peptide may be derived from a T15 HD peptide, a R24 HD peptide, a reverse T15 HD peptide, a reverse R24 HD peptide or conserved variants thereof. For example, the first and second HD peptide of the dimer may comprises an amino acid sequence having at least 90% sequence identity to SEQ ID No. 5, 7, 11, 12 or 13.

The HD peptides within the dimer may be joined by a linker. In some embodiments, the linker may be gly-gly.

The present disclosure further provides fusion proteins (e.g. chemically conjugated or recombinant antibodies) comprising the HD peptides and/or HD dimers as described herewith. The fusion proteins may comprise an immunoglobulin component or antibody and the HD peptide or HD peptide dimer may be recombinant or the HD peptide or HD peptide dimer may be conjugated thereto, in a manner that does not disrupt antigen binding and that allows preferred conformation changes in the HD peptide sequence thereby imparting dimerization activity. The HD peptides and HD peptide dimers disclosed herein may be used to enhance the potency of therapeutic antibodies or to increase binding sensitivity and/or avidity for other application such as diagnostics.

The term “homologous dimerization”, also referred to as “authophilic”, describes an entity that is self-associating. For example, “homodimerizing antibodies” or “autophilic antibody” are antibodies that self-bind. The term “homologous dimerization (HD) peptide” or “autophilic peptide” is a peptide that enables self-association or self-binding e.g. of an antibody or other immunoglobulin.

The term “naturally occurring HD peptide” or “native HD peptide” refers to an HD peptide found in nature. For example, “T15” refers to an anti-phosphorylcholine antibody and the T15 HD peptide refers to the HD peptide found in the T15 antibody having the amino acid sequence SRNKANDYTTEYSASVKGRFIVSR (SEQ ID No. 1). “R24” refers to an antibody recognizing disialoganglioside GD3 (J. Biol. Chem 374:5597-55604, 1999) and the R24 HD peptide refers to the HD peptide found in the R24 antibody having the amino acid sequence VAYISSGGSSINYA (SEQ ID No. 3).

The term “reverse configuration” or “reverse sequence” in relation to an HD peptide sequence means that the amino acid sequence of the naturally occurring HD peptide is in reverse order, i.e. the N-terminus becomes the C-terminus and the C-terminus becomes the N-terminus. The reverse sequence may be obtained by reading a peptide or protein sequence backward. The backwardly read sequence (retro sequence) is a new peptide (retro peptide) or protein sequence (retro protein).

The term “antibody” refers generally to a heavy or light chain immunoglobulin molecule or any functional combination or fragment thereof containing an antigen binding site, e.g. an antigen-binding fragment (Fab). The antibody is preferably specific for a cellular receptor, on a membrane structure such as a protein, glycoprotein, polysaccharide or carbohydrate, and on a normal cell or on tumor cells. The antibody may be a full-length immunoglobulin molecule or a variable domain fragment of an antibody. The term “antibody” encompasses nanobodies, bi-specific antibodies and diabodies, Fv and Fab, F(ab)₂, camelid and other antigen-binding scaffolds.

The term “chimeric” refers to a combination of components from different genetic sources or species. For example a chimeric antibody according to the present disclosure may comprise an immunoglobulin portion from one antibody and an HD peptide as described herein. A chimeric antibody may also include immunoglobulin portions derived from two or more sources or species and an HD peptide.

A “conservative variant” with reference to an amino acid sequence refers to a variant of the amino acid sequence with one or more conservative substitutions.

As used herein, the term “conserved substitution” or “conservative substitution” and grammatical variations thereof, refers to the presence of an amino acid residue in the sequence of the HD peptide that is different from, but is in the same class of amino acid as the described substitution or described residue (i.e., a nonpolar residue replacing a nonpolar residue, an aromatic residue replacing an aromatic residue, a polar-uncharged residue replacing a polar-uncharged residue, a charged residue replacing a charged residue). In addition, conservative substitutions can encompass a residue having an interfacial hydropathy value of the same sign and generally of similar magnitude as the residue that is replacing the wildtype residue.

As used herein, the term “nonpolar residue” refers to glycine (G, Gly), alanine (A, Ala), valine (V, Val), leucine (L, Leu), isoleucine (I, Ile), and proline (P, Pro): the term “aromatic residue” refers to phenylalanine (F, Phe), tyrosine (Y, Tyr), and tryptophan (W, Trp); the term “polar uncharged residue” refers to serine (S, Ser), threonine (T, Thr), cysteine (C, Cys), methionine (M, Met), asparagine (N, Asn) and glutamine (Q, Gln); the term “charged residue” refers to the negatively charged amino acids aspartic acid (D, Asp) and glutamic acid (E, Glu), as well as the positively charged amino acids lysine (K, Lys), arginine (R, Arg), and histidine (H, His). Other classification of amino acids may be as follows:
amino acids with hydrophobic side chain (aliphatic): Alanine (A, Ala), Isoleucine (I, Ile), Leucine (L, Leu), Methionine (M, Met) and Valine (V, Val):
amino acids with hydrophobic side chain (aromatic): Phenylalanine (F, Phe), Tryptophan (W, Trp), Tyrosine (Y, Tyr);
amino acids with polar neutral side chain: Asparagine (N, Asn), Cysteine (C, Cys), Glutamine (Q, Gln), Serine (S, Ser) and Threonine (T, Thr):
amino acids with electrically charged side chains (acidic): Aspartic acid (D, Asp), Glutamic acid (E, Glu):
amino acids with electrically charged side chains (basic): Arginine (R, Arg); Histidine (H, His): Lysine (K, Lys), Glycine G, Gly) and Proline (P, Pro).

Conservative amino acid substitutions are likely to have a similar effect on the activity of the resultant HA protein variant or modified HA protein, as the original substitution or modification. Further information about conservative substitutions can be found, for instance, in Ben Bassat et al. (J. Bacteriol, 169:751-757, 1987), O'Regan et al. (Gene, 77:237-251, 1989), Sahin-Toth et al. (Protein ScL, 3:240-247, 1994), Hochuli et al (Bio/Technology, 6:1321-1325, 1988) and in widely used textbooks of genetics and molecular biology.

The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff et al., Proc. Natl. Acad. Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected. The following table shows exemplary conservative amino acid substitutions: Table 1.

TABLE 1
Exemplary conservative amino acid substitutions.

| | Very Highly - | Highly Conserved | |
|---|---|---|---|
| Original | Conserved | Substitutions (from the | Conserved Substitutions |
| Residue | Substitutions | Blosum90 Matrix) | (from the Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, Ser, Thr | Arg, Asp, Gln, Glu, His, Lys, Ser, Thr |
| Asp | Glu | Asn, Glu | Asn, Gin, Glu, Ser |
| Cys | Ser | None | Ala |
| Gin | Asn | Arg, Asn, Glu, His, Lys, Met | Arg, Asn, Asp, Glu, His, Lys, Met, Ser |
| Glu | Asp | Asp, Gln, Lys | Arg, Asn. Asp, Gln, His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | lle, Leu, Met, Trp, Tyr |
| Ser | Thr | Ala, Asn, Thr | Ala, Asn, Asp, Gln, Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

Without wishing to be bound by theory, it is believed that by increasing hydropathy (by using hydropathic indices for individual amino acids) the level of binding might be increased, whereas reduction of hydropathy may decrease self-binding.

In some embodiments, the HD peptide sequences may be modified to enhance the crosslinking potential of the HD antibodies as described herein. In one embodiment, such functionally enhanced peptides are determined by producing a series of synthetic peptides with substitutions at each amino acid position within the template sequence and then testing this library of peptides for autophilic binding or for binding to the original peptide sequence. Those peptides with superior binding to the original sequence are then conjugated to immunoglobulins and the resultant conjugates are tested for potency, specificity, and the unwanted ability to induce aggregation. In one specific embodiment, the T15 peptide sequence is altered and modified sequences are selected for enhanced function. In another embodiment of the invention, the self-binding potential of a peptide can be enhanced by increasing complementarity of the sequence, such as described in U.S. Pat. No. 4,863,857 (issued to Blalock et al.). The self-binding potential and/or toleration of a peptide can also be enhanced by humanizing a self-binding peptide sequence derived from non-human animals. Humanizing a peptide sequence involves optimizing the sequence for expression or functionality in humans. Examples and methods of humanizing peptides and proteins have been described elsewhere (Roque-Navarro et al., 2003; Caldas et al., 2003: Leger et al., 1997; Isaacs and Waldmann, 1994: Miles et al. 1989; Veeraraghavan et al., 2004; Dean et al., 2004: Hakenberg et al., 2003: Gonzales et al., 2004; and H. Schellekens, 2002).

The term “expression construct” refers to a recombinant nucleic acid sequence including a nucleic acid sequence encoding a peptide or protein to be expressed. The nucleic acid encoding a peptide or protein to be expressed is operably linked to one or more regulatory nucleic acid sequences that facilitate expression of the peptide or protein to be expressed. Nucleic acid sequences are operably linked when they are in functional relationship. A regulatory nucleic acid sequence is illustratively a promoter, an enhancer, a DNA and/or RNA polymerase binding site, a ribosomal binding site, a polyadenylation signal, a transcription start site, a transcription termination site or an internal ribosome entry site (IRES). An expression construct can be incorporated into a vector, such as an expression vector and/or cloning vector. The term “vector” refers to a recombinant nucleic acid vehicle for transfer of a nucleic acid. Exemplary vectors are plasmids, cosmids, viruses and bacteriophages. Particular vectors are known in the art and one of skill in the art will recognize an appropriate vector for a specific purpose.

### Homologous Dimerization (HD) Peptides

The present application describes homologous dimerization (HD) peptides with improved self-binding and is exemplified with T15 and R24 HD peptides (see Example 1). Analysis of the self-binding and hydropathic character of the T15 peptide and other HD peptides produced a motif useful in predicting amino acid substitutions that may lead to improved self-binding.

Exemplary homologous dimerization peptides described herein are summarized in Table 2 below.

TABLE 2
autophilic/homologous dimerization (HD) sequences

| SEQ | | | |
|---|---|---|---|
| ID | | | |
| NO. | HD peptide name | Derivation | Sequence |
| 1 | T15 | T15 anti-phosphorylcholine | ASRNKANDYTTEYSASVKGRFIVSR* |
| 2 | Anti-viral peptide | CR3014 anti-SARS | RIRNQAYSYTTEYAASVKGRFTISR** |
| 3 | R24 (short) | R24 anti-disialoganglioside | VAYISSGGSSINYA |
| 4 | rs-T15 | T15 anti-phosphorylcholine | RSVIFRGKVSASYETTYDNAKNRSA |
| 5 | rs-T15 dimer (rs- | T15 anti-phosphorylcholine | RSVIFRGKVSASYETTYDNAKNRSAGGRSVIFRGKVSASYETTYDNAKNRSA |
| | GG-rs) | | |
| 6 | rs-R24 | R24 anti-disialoganglioside | AYNISSGGSSIYAY |
| 7 | rs-R24 dimer (rs- | R24 anti-disialoganglioside | AYNISSGGSSIYAYGGAYNISSGGSSIYAY |
| | GG-rs) | | |
| 8 | T15-var1 | T15 anti-phosphorylcholine | ASRKKANDYTTEYSASVKGRFIVSR |
| 9 | T15-var2 | T15 anti-phosphorylcholine | ASRNKARDYTTEYSASVKGRFIVSR |
| 10 | T15-var3 | T15 anti-phosphorylcholine | ASRNKANDYTTEYSASVHGRFIVSR |
| 11 | T15-var1 dimer | T15 anti-phosphorylcholine | ASRKKANDYTTEYSASVKGRFIVSRGGASRKKANDYTTEYSASVKGRFIVSR |
| 12 | T15-var2 dimer | T15 anti-phosphorylcholine | ASRNKARDYTTEYSASVKGRFIVSRGGASRNKARDYTTEYSASVKGRFIVSR |
| 13 | T15-var3 dimer | T15 anti-phosphorylcholine | ASRNKANDYTTEYSASVHGRFIVSRGGASRNKANDYTTEYSASVHGRFIVSR |
| 14 | T15 dimer | | ASRNKANDYTTEYSASVKGRFIVSRGGASRNKANDYTTEYSASVKGRFIVSR |
| 15 | R24 dimer (short) | | VAYISSGGSSINYAGGVAYISSGGSSINYA |
| 16 | R24 (long) | | GAAVAYISSGGSSINYA |
| 17 | R24 dimer (long) | | GAAVAYISSGGSSINYAGGAAVAYISSGGSSINYA |
| 18 | rs-R24 (long) | | AYNISSGGSSIYAVAAG |
| 19 | Rs-R24 dimer | | AYNISSGGSSIYAVAAGGAYNISSGGSSIYAVAAG |
| | (long) | | |

*YTTEY (bold in SEQ ID NO: 1) denotes the potential hinge region
**SEQ ID 2 is a putative natural self-binding antibodies. The sequences may be enhanced by the motif of hydropathy as disclosed in the current disclosure

A substantially identical amino acid sequence of an immunoglobulin component has an amino acid sequence at least 70%, 80%, 85%, 90% and more preferably 95%, 96%, 97%, 98%, 99% or greater % identical to an amino acid sequence disclosed herein in particular embodiments of the present invention, wherein the substantially identical protein retains a substantially similar or better function compared to the reference protein with which it is substantially identical. As will be appreciated by one of skill in the art, the degeneracy of the genetic code is such that more than one nucleic acid will encode a particular immunoglobulin component and these alternative sequences are considered within the scope of the present invention.

An amino acid sequence which is substantially identical to the 25-mers of SEQ ID Nos. 4 and 8-10 has at least 20 contiguous amino acids, more preferably at least 22 contiguous amino acids, having an amino acid sequence at least 70%, 80%, 85%, 90% and more preferably 95%, 96%, 97%, 98%, 99% or 100% identical to 20 or more contiguous amino acids of the identified autophilic amino acid sequence. An amino acid sequence which is substantially identical to the 14-mers of SEQ ID Nos. 3 and 6 has at least 10 contiguous amino acids, more preferably at least 8 contiguous amino acids, having an amino acid sequence at least 70%, 80%, 85%, 90% and more preferably 95%, 96%, 97%, 98%, 99% or 100% identical to 10 or more contiguous amino acids of the identified autophilic amino acid sequence. The same applies to each amino acid sequence in the HD-dimers e.g. SEQ ID Nos. 5, 7 11, 12 and 13. The linker does not necessarily need to be gly-gly and may be any other suitable linker.

An amino acid sequence having an amino acid sequence at least 70%, 80%, 85%, 90% and more preferably 95%, 96%, 97%, 98%, 99% or 100% or any amount therebetween identity or similarity to the sequence of SEQ ID NOs 1, 2, 3, 4, 5, 6, 7, 8, 9, 10, 11, 12, 13, 14, 15, 16, 17, 18, or 19.

The substantially identical amino acid sequences may comprise one or more conservative substitution and may be referred to as a conservative variant. A peptide which is substantially identical to a HD peptide described herein retains a substantially similar or better autophilic function compared to the reference autophilic peptide with which it is substantially identical.

### Homodimerizing Antibodies (Also Referred to as Autophillic, Self-Binding Antibodies or HD Antibodies)

The present disclosure describes the generation of an antibody-peptide fusion protein that enhances the biological and immunological activity of the antibody without changing the antibody specificity for the corresponding antigen. Specifically, the present disclosure provides the generation of antibody fusion proteins containing the complete or partial autophilic 24 mer peptide derived from T15 or the 17-mer peptide derived from R24. The HD peptides may be provided in reverse configuration, as dimers or with hydrophilic substitutions.

CDRs from any antibody may be used in the peptide/antibody complex as described herewith. Preferred antibodies are ones binding immuno-regulatory or checkpoint inhibitors. For example the CDR sequences may encode for antibodies specific for PD-1/PD-1L or CTLA-4 and expressing activity for T-cell activation. Any restrictions on peptide length are those practical limitations associated with peptide synthesis and not restrictions associated with practice of the method of the disclosure. Other preferred CDR sequences originate from FDA approved antibodies such as Rituxin, Herceptin and/or Avastin.

Recombinant monoclonal antibodies have been created with the genes for CDRs (antigen binding sequences) of non-human species (typically mouse) inserted into a human antibody framework for the primary purpose of decreasing immunogenicity. Although these recombinant antibodies have the ability to interact with effector cells and human complement, therapeutic activity, if any, is usually imparted from the original CDR sequences due to the nature of the antigen/antibody interaction.

HD peptides, attached to antibodies, allow for the formation of lattices at the cell surface incorporating both antibody bound to target as well as antibody bound only by self-binding interactions with other target bound antibodies. This not only allows for lattice formation and crosslinking of cell surface antigen but also allows for higher than the typical 1:1 ratio of antibody bound to antigen epitopes. Based on flow cytometry analysis this ratio, at least in some antigen-antibody systems, can be as high as 50-100 fold. In fact, this greatly increased binding of an HD modified antibody, is the hallmark of HD antibodies and is used as the initial assay for assessment of recombinant and chemically-conjugated antibodies.

The discovery of homologous dimerization sequences (HD) that allow antibodies to self-associate, form lattices and crosslink a target antigen offer the opportunity to impart therapeutic activity to a recombinant monoclonal antibody that is independent of the antigen binding CDR sequences and therefore applicable to many antigen/antibody systems. Such peptide sequences have been previously chemically conjugated to antibodies through the nucleotide binding site and inserted on the C-terminus of the Fc region of a fusion protein. The former methodology allows insertion of the HD peptide into the antibody in a site specific manner but is not a scaleable manufacturing process. The latter approach in contrast is scaleable but does not allow for optimal activity. For optimal HD activity in antibodies, the peptide needs to interact at multiple amino acid positions with a HD peptide on an adjacent antibody. This self-binding can be reduced by the peptide's tendency to form a hairpin structure near the c-terminus of the HD peptide. Unexpectantly, it was found that HD peptide sequences could be inserted into recombinant antibodies or by site-specific conjugation without loss of antigen binding or reduction in effector functions by careful choice as to the position of the peptide, e.g. by reversing the order of the amino acids in the HD peptide. The problem of loss of HD activity upon insertion onto the C-terminus of the Fc region, the previous recombinant antibody approach, was overcome by reversing the normal sequence order (N-terminus to c-terminus) not allowing for the C-terminus of the HD peptide to form its preferred hairpin structure. Similar results could also be achieved by dimerizing the HD peptide either in the forward or reverse configuration, and linking the two HD peptides with a linker such as gly-gly.

Incorporating HD technology into other antibody forms other than the prototypical human or humanized IgG1 antibody form will improve their therapeutic or diagnostic activity due to greatly enhanced binding. Such antibody forms include but are not limited by nanobodies, bi-specific antibodies and diabodies, Fv and Fab, F(ab)2, camelid and other antigen-binding scaffolds (Reviewed in: Hoglan Yu, Abhiskek Saxena, Sachdev S. Sidhu, Donghui Wu, Frontiers of Immunology 8: Article 38, 2017).

A group of recombinant antibody forms, modified within the Fc region, to increase or decrease serum half-life, antibody-dependent cellular cytotoxicity, complement binding or complement-mediated killing also represent antibody forms for which increased antigen binding through HD technology can enhance therapeutic or diagnostic activity (Reviewed in: Abhishek Saxena & Donghui Wu, Frontiers in Immunology, 7: Article 580, 2016). The recombinant antibody forms of the present invention are not limited by these referenced forms.

Antibodies according to the present invention can spontaneously bind to self only after first binding to their target antigen. The homodimerizing antibodies of the present invention preferably bond non-covalently with other such conjugated antibodies when bound to their target antigen(s), usually a cell-surface, trans-membrane receptor(s).

Homodimerizing antibodies of the present invention typically comprise antibodies conjugated with one or more peptides having an HD peptide sequence. A homodimerizing antibody of the invention can comprise virtually any immunoglobulin. In some embodiments, the antibodies bind to targets implicated in a disease or disorder, where binding of the target has a therapeutic effect on the disease or disorder. The target antigens can include cell-surface antigens, including trans-membrane receptors. In specific embodiments, the Ig component of the antibodies can comprise a monoclonal antibody.

The present invention affords antibodies having self-binding properties that mimic those of rare, naturally occurring, autophilic antibodies. The invention thereby offers a simple and attractive alternative to covalent dimerization and other engineering approaches directed to enhancing the therapeutic potential of antibodies.

### Expression Systems

The invention provides an isolated host cell transformed with an expression vector encoding an immunoglobulin heavy chain having an antigen binding domain and an HD peptide. In particular embodiments, the isolated host cell is also transformed with an expression vector encoding an immunoglobulin light chain having an antigen binding domain and the antigen binding domain of the immunoglobulin heavy chain and the antigen binding domain of the immunoglobulin light chain together form an antigen binding site. The isolated host cell for producing a recombinant autophilic antibody of the present invention may be in vitro. Expression systems for HD antibody expression illustratively include: eukaryotic cells such as mammalian cells, plant cells, insect cells, yeast, and amphibian cells: and prokaryotic expression systems such as bacteria. One of skill in the art is able to select a particular expression system for use in producing a recombinant HD antibody.

### Location of HD Peptide

As seen in US Patent Publication No. 20030103984, it was previously only considered practical to insert the HD peptide sequence on the c-terminus of the Fc region. This was due to the concern that insertion of the HD peptide in other regions of the mAb might reduce or disrupt antigen binding or potential therapeutic activities such as ADCC or complement binding. In fact, published data indicates that the 24 mer HD sequence derived from the T15 antibody when synthesized and assayed expresses three-dimensional conformation: encoding it into an antibody might not only negate that 3D structure of the peptide but also may impair antigen binding of nearby CDRs. Although self-associating activity in the Fc terminus construct (Kohler, H, Rector, K & Amick J., Hybridoma 2012 (6): 395-402) was described in comparison to a nucleotide affinity site chemical conjugate, the activity was reduced (data not shown).

In preferred embodiments, of the invention, the HD peptide is localized to one of three positions within the antibody, e.g. IgG.

The first is immediately following the CDR3 of the heavy chain or light chain. In this form, the Ig molecule can be expressed as a single chain Fv with the HD peptide encoded in any of the optimized configurations described herein on the most c-terminal portion preceded by a gly-gly or similar spacer. Despite the proximity to the antigen binding portion of the antibody this location can impart maximal self-association without a reduction in antigen binding. The retention of antigen binding in this location was totally unexpected, as we, the inventors of the original technology considered this so improbable that we actually previously linked the sequence onto the c-terminus of the Fc region of the whole IgG—the furthest we could separate antigen binding from self-association.

A second site is immediately c-terminal to the heavy or light chain constant region preceded by a gly-gly spacer. This is can be expressed as a Fab. In this form, the Fab can also be part of a naïve or immunized phage expression library assayed for binding to a target antigen. Using the HD peptide would allow for enhanced binding and identification of even low affinity antibodies.

The third site is one in which the HD sequence is cloned onto the c-terminus of the Fc region preceded by a gly-gly or other spacer. In embodiments where the HD peptide is a dimer of an HD peptide, in normal or reverse configuration, the separation of the two HD peptides by a gly-gly or alternative spacer is especially relevant to the antibody c-terminus constructs to overcome steric inhibition. In initial testing this form of the HD peptide has a higher avidity of interaction.

### Methods of Producing Homodimerizing (Autophilic) Antibodies

HD peptide modified antibodies created by a variety of site-specific chemical conjugation methods can also be used to create fully active, self binding antibodies. Numerous methods for attaching HD peptides to antibody molecules will be known by those skilled in the art. One method is to use chemical crosslinking, such as the affinity-crosslinking method described in US Patent Publication No. 20040185039. Such a method was initially adopted due to the fact that it is able to link a peptide into the nucleotide binding site of antibodies which is localized to the end of the heavy chain of the Fab. This affinity site allows insertion of the peptide without reducing affinity of binding and retains the self-association activity of the peptide. When compared to conjugation of HD-peptide to carbohydrate, such site-specific conjugates near the antigen-binding region were more active, even though conjugation through carbohydrate resulted in more peptide bound to antibody (J. Immunol Methods: (2005) 304:100-106, Photo-activated affinity-site cross-linking of antibodies using tryptophan containing peptides, Mike Russ, Dingyuan Lou, Heinz Kohler). However, due to elements of the methodology, namely the photoactivation step, the manufacturing process for binding for this method is not scaleable and only useful to produce small quantities of conjugate.

A second method of site specific conjugation has been developed for use with antibody-drug conjugates (ADC), namely Smartag technology (Wu, P., et al., Site-specific chemical modification of recombinant proteins produced in mammalian cells by using for genetically encoded aldehyde tag. Proc Natl Acad Sci U S A, 2009. 106 (9): p. 3000-5.). Use of such a technology or similar site specific technologies with HD peptides could overcome the issue of scaleability.

Alternatively, recombinant methods may be used. For example, a fusion gene comprising a nucleic acid sequence encoding an antibody and a nucleic acid sequence encoding the peptide may be prepared, wherein the nucleic acid sequence encoding the peptide is located inside the nucleic acid sequence encoding the antibody at a site wherein, when the fusion is expressed, the fusion protein that is created thereby includes the antibody plus the peptide, and the peptide is connected to the antibody at a site that does not interfere with antigen binding of the antibody, and expressing the fusion gene to create the fusion protein. In particular, the fusion protein may be created by providing a gene encoding an antibody, wherein the gene is mutated to contain a restriction site, wherein the restriction site is located immediately C-terminal to the CDR3 of the antibody, or immediately c-terminal to the heavy chain variable region or onto the c-terminus of the antibody. A humanized antibody generated by CDR swapping of mouse or other human or non-human sources together with a human IgG framework encoding an HD-peptide may be expressed by the fusion gene.

Methods of creating fusion proteins are described, for example, in the following U.S. patents: U.S. Pat. No. 5,563,046 to Mascarenhas et al; U.S. Pat. No. 5,645,835 to Fell, Jr.; U.S. Pat. No. 5,668,225 to Murphy; U.S. Pat. No. 5,698,679 to Nemazee; U.S. Pat. No. 5,763,733 to Whitlow et al; U.S. Pat. No. 5,811,265 to Quertermous et al; U.S. Pat. No. 5,908,626 to Chang et al; U.S. Pat. No. 5,969,109 to Bona et al; U.S. Pat. No. 6,008,319 to Epstein et al; U.S. Pat. No. 6,117,656 to Seed; U.S. Pat. No. 6,121,424 to Whitlow et al; U.S. Pat. No. 6,132,992 to Ledbetter et al; U.S. Pat. No. 6,207,804 to Huston et al; and U.S. Pat. No. 6,224,870 to Segal. Methods of creating Ig fusion proteins are described, for example, in Antibody Engineering, 2nd Edition. ed.: Carl A. K. Borrebaeck, Oxford University Press 1995, and in “Molecular Cloning: A Laboratory Manual, Second Ed., Cold Spring Harbor Press, 1989.

In specific embodiments, a DNA sequence encoding an HD peptide described herein, or a substantially identical HD peptide is inserted in-frame with a DNA sequence encoding an immunoglobulin heavy chain and/or immunoglobulin light chain. The fusion protein (or HD antibody) expressed from the DNA sequence contains an immunoglobulin heavy chain and/or immunoglobulin light chain having said HD peptide.

Nucleic acids encoding immunoglobulin heavy chains or immunoglobulin light chains are well-known and any of various nucleic acids encoding immunoglobulin heavy chains or immunoglobulin light chains can be used to produce a recombinant chimeric HD antibody of the present invention. Specific nucleic acids are described herein which encode human constant heavy chain and/or a human constant light chains, particularly human gamma constant heavy chains and human kappa constant light chains. Nucleic acids encoding human gamma constant heavy chains and/or human kappa constant light chains can be obtained from commercial sources, such as vector pAc-k-CH3, available from Progen Biotechnik GmbH. Nucleic acids encoding protein and/or peptides described herein, including human gamma constant heavy chains and/or human kappa constant light chains, can be produced using recombinant techniques such as by cloning or synthesis. Particular immunoglobulin constant heavy chains and/or immunoglobulin kappa constant light chains, are described, for instance, in U.S. Pat. Nos. 5,736,137 and 6, 194,551.

### IgG Fusion Proteins

Ig fusion proteins have the advantage of joining the antibody combining specificity and/or antibody effector functions with molecules contributing unique properties. The ability to produce this family of proteins was first demonstrated when c-myc was substituted for the Fc of the antibody molecule, (Neuberger M S, Williams G T and Fox R O. Nature 125:604, 1984) but many examples now exist. Ab fusion proteins can be achieved in several different ways. In one approach non-Ig sequences are substituted for the variable region; the molecule replacing the V region provides specificity of targeting with the antibody contributing properties such as effector functions and improved pharmacokinetics. Examples include IL-2 and CD4. Alternatively, non-Ig sequences can be substituted for or attached to the constant region. The resulting molecules retain the binding specificity of the original antibody but gain characteristics from the attached protein. Depending on the position of the substitution, different antibody-related effector functions and biologic properties will be retained.

### Vectors for the Construction of IgG Fusion Proteins

A series of vectors have been produced that permits the fusion of proteins at different positions within an antibody molecule, thereby facilitating the construction of fusion proteins with different properties. Using these vectors it is possible to produce a family of fusion proteins with molecules of differing molecular weight, valence, and having different subsets of the functional properties of the antibody molecule.

As a specific example of how to facilitate the construction of fused genes, site-directed mutagenesis was used to generate unique restriction enzyme sites in the human IgG3 heavy chain gene. In this particular example, restriction sites were generated at the 3′ end of the CH1 exon, immediately after the hinge at the 5′ end of the CH2 exon, and at the 3′ end of the CH3 exon. The restriction sites thus produced were SnaB I at the end of CH1 by replacing TtgGTg with TacGTa, Pvu II at the beginning of CH2 by replacing CAcCTG with CAgCTG, and Ssp I at the end of CH3 replacing AATgag with AATatt. These manipulations provided a unique blunt-end cloning site at these positions. In all cases the restriction site was positioned so that after cleavage the Ig would contribute the first base of the codon. Human IgG3 with an extended hinge region of 62 amino acids was chosen for use as the immunoglobulin; when present this hinge should provide spacing and flexibility, thereby facilitating simultaneous antigen and receptor binding. An EcoR I site was also introduced at 3′ of the IgG3 gene to provide a 3′ cloning site and polyA addition signal. Although initially designed for use with growth factors, these restrictions sites can be used to position any novel sequence at defined positions in the antibody. Also, using these cloning cassettes the variable region can easily be changed. Similar techniques may be used to generate suitable restriction sites in other antibody genes.

### Production of A Fusion Gene

As a first step in the production of a fusion protein, a blunt-end restriction site must be introduced at the desired position into the 5′ end of the gene to be fused. In order to maintain the correct reading frame, the site must be positioned so that after cleavage it will contribute two bases to the codon. If the objective is to make a fusion protein with the complete molecule, the restriction site is usually introduced at the position of any post-translational processing, such as after the leader sequence. Alternatively, if the objective is to use only a portion of the protein, the blunt-end site can be introduced at any position within the gene, but attention must always be paid to maintaining the correct reading frame. Additionally, if there is carboxyl-terminal post-translational processing of the fused protein, it is frequently desirable to introduce a stop-codon at this processing site.

A major concern when producing fusion proteins is maintaining the biologic activities of all of the components. The production of fusion proteins with antibodies is facilitated by the domain structure of the antibody, and all of the cloning sites have been positioned immediately following an intact domain. In this configuration the correct folding of the immunoglobulin should be assured. The folding of the attached protein depends on its structure and where it is fused. Whenever structural information is available, it is desirable to produce the fusion at a position that will maintain the structural integrity of the attached protein.

To produce quantities of protein sufficient for functional analysis, it is desirable to have the protein secreted into the medium. While in the examples reported to date, assembled fusion proteins have been assembled and secreted, this remains a concern when designing additional fusion proteins.

The method to design a fusion gene that contains a biologically activity peptide as part of the heavy or light chain gene can use established antibody engineering protocols (Antibody Engineering, 2nd Edition. ed.: Carl A. K. Borrebaeck, Oxford University Press 1995. Chapter 9, pages 267-293). The peptide can fused either to N-terminal residues or the C-terminal residues of H or L chains. The expression of such fused genes is typically done in mammalian cell lines, although other expression systems, such as, for example, bacteria or yeast expression systems, may be used.

### Pharmaceutical Compositions

The invention also relates to compositions comprising a homodimerizing antibody of the invention and a pharmaceutically acceptable carrier.

The antibodies of the invention are useful in pharmaceutical compositions for systemic administration to humans and animals in unit dosage forms, sterile solutions or suspensions, sterile non-parenteral solutions or suspensions oral solutions or suspensions, oil in water or water in oil emulsions and the like, containing suitable quantities of an active ingredient. Topical application can be in the form of ointments, creams, lotions, jellies, sprays, douches, and the like. The compositions are useful in pharmaceutical compositions (wt %) of the active ingredient with a carrier or vehicle in the composition in about 1 to 20% and preferably about 5 to 15%.

The above parenteral solutions or suspensions may be administered transdermally and, if desired a more concentrated slow release form may be administered. The fusion proteins of the invention may be administered intravenously, intramuscularly, intraperitoneally or topically. Accordingly, incorporation of the active compounds in a slow release matrix may be implemented for administering transdermally. The pharmaceutical carriers acceptable for the purpose of this invention are the art known carriers that do not adversely affect the drug, the host, or the material comprising the drug delivery device. The carrier may also contain preserving, stabilizing, wetting, emulsifying agents and the like together with the penetration enhancer of this invention. The effective dosage for mammals may vary due to such factors as age, weight activity level or condition of the subject being treated. Typically, an effective dosage of a compound according to the present invention is about 10 to 500 mg, when administered in solution at least once daily. Administration may be repeated at suitable intervals.

Conjugate autophilic antibodies can bind non-covalently with other autophilic antibodies when bound to their target antigen(s). However, premature formation of dimers or multimers of the antibodies may lead to difficulties in manufacturing, such as during purification and concentration, as well as drawbacks in administration, which may lead to side effects. As such, compositions containing autophilic antibody-peptide conjugates of the invention are formulated to reduce this dimerizing potential and maximize monomeric properties while in solution and before administration. For example, it has been found that solution dimerization can be reduced or mitigated by using a hypertonic composition. In some embodiments, salt concentrations of 0.5M or more, low levels of SDS or other various detergents such as those of an anionic nature (see U.S. Pat. No. 5,151,266, which is incorporated herein by reference), or modifications of the antibody to decrease its isoelectric point, for example through the use of succinyl anhydride (see U.S. Pat. No. 5,322,678, which is incorporated herein by reference), an be used to formulate compositions.

### Immunoassays

Immunoassays are provided according to the present invention which include contacting an analyte in a biological or environmental sample with an antibody conjugated to an autophilic peptide. A complex formed by the analyte and the antibody conjugated to an autophilic peptide is then detected.

In particular embodiments, assays of the present invention are characterized by detection of antigens expressed at low levels, such as on a cell surface, using antibodies containing autophilic peptides either naturally or by conjugation of an autophilic peptide to the antibody.

The use of highly-specific antibodies is common in many diagnostic applications. The binding of said antibodies may be detected directly by a number of means or, alternatively, secondary antibodies are required for signal enhancement and detection. Previously, the signal detection was directly related to the amount of antibody bound, either mono or divalently bound to target. In the present invention, enhanced signal strength results from polyvalent binding interactions of autophilic peptide-conjugated antibodies bound to a target analyte.

Whether directly or indirectly detected, autophilic peptide-conjugated antibodies greatly enhance signal detection because of lattice formation and more antibody surrounding the target. Thus, naturally occurring autophilic antibodies and non-naturally occurring autophilic peptide-conjugated antibodies can be directly labeled with a detectable label affording an enhanced signal in an immunoassay. Similarly, indirect labeling, such as labeling of a secondary antibody produces increased signal in an immunoassay since the secondary antibody will bind with increased numbers to the primary autophilic peptide-conjugated antibodies.

In embodiments of assays of the present invention, a naturally-occurring autophilic antibody or an antibody conjugated to a homologous dimerization (HD) peptide is used to enhance signal detection of antigen immobilized on a substrate, such as plastic, in assays such as ELISA.

In embodiments of assays of the present invention, a naturally-occurring autophilic antibody or an antibody conjugated to a homologous dimerization (HD) peptide is used to enhance “off-rate” or increase in avidity of antigen coated or bound to a polymer chip and detected by surface plasmon resonance.

In embodiments of assays of the present invention, a naturally-occurring autophilic antibody or an antibody conjugated to homologous dimerization (HD) peptide is used to enhance binding and localization detection of the antibody bound to a target in vivo, such as in a xenograft tumor animal model, by fluorescence or other signal detection method.

Assays according to embodiments of the present invention can include virtually any immunoglobulin conjugated with one or more homologous dimerization (HD) peptides for enhanced detection of an analyte.

The term “analyte” refers to any molecule or compound which is specifically recognized by an antibody conjugated to an autophilic peptide, illustratively including a protein, a peptide, a hapten, a carbohydrate, a lipid, a ganglioside and combinations of these. In particular embodiments, an analyte can be a mammalian analyte, illustratively including a protein, peptide, hapten, carbohydrate, lipid, ganglioside or combination thereof generated, for instance, by a normal or abnormal cell of a mammal.

In further particular embodiments, an analyte can be a non-mammalian analyte, such as a protein, peptide, hapten, carbohydrate, lipid, ganglioside or combination thereof, generated by a microorganism, such as a bacterium or a virus. Thus, particular assays of the present invention are provided for detection of a microorganism and/or a product of a microorganism. Assays for detection of a microorganism and/or a product of a microorganism are used to assay a sample obtained from a human or a non-human animal to detect infection, for example. In further embodiments, assays for detection of a microorganism and/or a product of a microorganism are used to assay a sample obtained from an environment or object to be tested for contamination with a microorganism and/or a product of a microorganism.

In embodiments of assays of the present invention, a target analyte is a B-cell receptor, CD20, Her2, ganglioside GM2, glycolic ganglioside GM3, GD3 ganglioside, caspases, oxidized low density lipoproteins, phosphocholine, EGFR, CD32B, HLADR1, CD19, EpCAM, PSA and bacterial antigens such as a staphylococcal antigens.

In some embodiments of the present invention, diagnostic and prognostic immunoassays are provided. The term “diagnostic immunoassay” refers to an immunoassay that allows for determination of presence or amount of an analyte indicative of a disease or pathological condition in an animal or human subject. The term “prognostic immunoassay” refers to an immunoassay that allows for determination of presence or amount of an analyte indicative of progression of a disease or pathological condition in an animal or human subject.

### Kits

In embodiments of the present invention, kits are provided for use in performing an assay using an antibody-homologous dimerization (HD) peptide conjugate. In particular embodiments, a kit includes an antibody-homologous dimerization (HD) peptide conjugate and instructions for use in detecting an analyte in a sample.

### Methods of treatment

A method of enhancing apoptosis, complement fixation, effector cell-mediated killing of targets, or preventing the development of, or enhancement of, a disease state, is also contemplated, which employs a homologous dimerization (HD)-antibody of the invention or a composition comprising the homologous dimerization (HD)-antibody. In one embodiment, an autophilic conjugate of the invention, or a composition containing an autophilic conjugate of the invention, is administered to a subject. Once administered, the antibodies bind to target cells and enhance apoptosis, complement fixation, effector cell-mediated killing of targets, or prevent target antigens or cells from stimulating the development of, or further enhancing, a disease state. In a further embodiment, allowing time for the autophilic conjugate to bind to target cells and enhance apoptosis, complement fixation, effector cell-mediated killing of targets, or prevent target antigens or cells from further enhancing a disease state, and for the autophilic conjugate to be cleared from normal tissues, a second anti-autophilic peptide antibody can be administered.

In some aspects, a patient who suffers from a debilitating or potentially life-threatening disease or condition is administered at least one subject homologous dimerization (HD) antibody in an amount effective to alleviate symptoms of the disease or condition. A disease or condition contemplated for treatment by an antibody of the invention can be a malignancy, neoplasm, cancer, auto-immune disorder, Alzheimer's disease or other neuro-degenerative condition, or graft or transplantation rejection.

In some aspects, a method of potentiating apoptosis of targeted cells of a patient comprises administering a first homologous dimerization (HD) antibody-peptide conjugate and a second antibody that recognizes the peptide domain of the conjugate. In this embodiment, the antibody-peptide conjugate recognizes the extracellular region of a transmembrane receptor of the target cell. Owing to its homodimerization property, the antibody-peptide conjugate can bind more avidly to the target than the corresponding antibody lacking the self-binding peptide domain. Moreover, whenever the autophilic antibodies bind to two or more receptors, with those receptors being brought in close proximity due to the self-binding property of the antibodies, an apoptosis signal within the cell can be triggered. In those instances when the peptide domain of the conjugate presents an exposed epitope, a second antibody, specific for the autophilic peptide, can be administered, bind to the modified antibody, and enhance the process of crosslinking and even cause temporary clearance of the target antigen. If the target antigen is a receptor, clearance from the cell surface, endocytosis, and degradation will subsequently require synthesis of new receptor protein, meaning that the biological function of the receptor will be more effectively inhibited for a longer period than using either a simple blocking antibody or small molecule inhibitor. Alternatively, the second antibody can bear a radiolabel or other potentially therapeutic substance, so that when administered it can attack the targeted cells. The key to use of this second antibody is that antibody's specificity. The homologous dimerization (HD) peptide, though naturally occurring, is present on only a small number of murine immunoglobulins. Thus, antibody specific to this peptide will have the requisite selectivity to be used in vivo.

### Diseases

A disease or condition contemplated for treatment by an antibody of the invention can be a malignancy, neoplasm, cancer, atherosclerosis, auto-immune disorder, Alzheimer's disease or other neurodegenerative condition, graft or transplantation rejection, or any other disease or condition responsive to antibody therapy.

### Doses

The homologous dimerization (HD) antibodies described herein can be administered in one or more dosage amounts substantially identical to or less than those practicable for unmodified antibodies.

TABLE 3
SEQ ID NO: and Description of Sequences

| SEQ ID NO: | Description of Sequence |
|---|---|
| 1 | T15 |
| 2 | CR3014 anti-SARS |
| 3 | R24 (short) |
| 4 | rs-T15 |
| 5 | rs-T15 dimer |
| 6 | rs-R24 |
| 7 | rs-R24 dimer |
| 8 | T15-var1 |
| 9 | T15-var2 |
| 10 | T15-var3 |
| 11 | T15-var1 dimer |
| 12 | T15-var2 dimer |
| 13 | T15-var3 dimer |
| 14 | T15 dimer |
| 15 | R24 dimer (short) |
| 16 | R24 (long) |
| 17 | R24 dimer (long) |
| 18 | Rs-R24 (long) |
| 19 | Rs-R24 dimer (long) |
| 20 | Rituximab heavy chain |
| 21 | Rituximab light chain |
| 22 | Herceptin Light chain (1 and 2) |
| 23 | Herceptin Heavy chain (1 and 2) |
| 24 | Bevacizumab light chain |
| 25 | Bevacizumab heavy chain |
| 26 | Atezolizumab (anti-PD-L1 mAb) heavy chain |
| 27 | Atezolizumab (anti-PD-L1 mAb) light chain |

The following examples are presented to illustrate certain aspects of the invention, and are not intended to limit the scope of the invention.

The present invention will be further illustrated in the following examples.

### EXAMPLES

Example 1—HD Peptides with Potential for Optimized Self-Binding

The 24 mer peptide sequence derived from T15 was analyzed and compared with other anti-phosphorylcholine antibodies of the same germline configuration that possess limited or no HD activity using hydropathic scores of individual amino acids and the overall peptide hydropathic plot (Kyte-Doolittle), a method of protein analysis normally employed with non-immunoglobulin proteins.

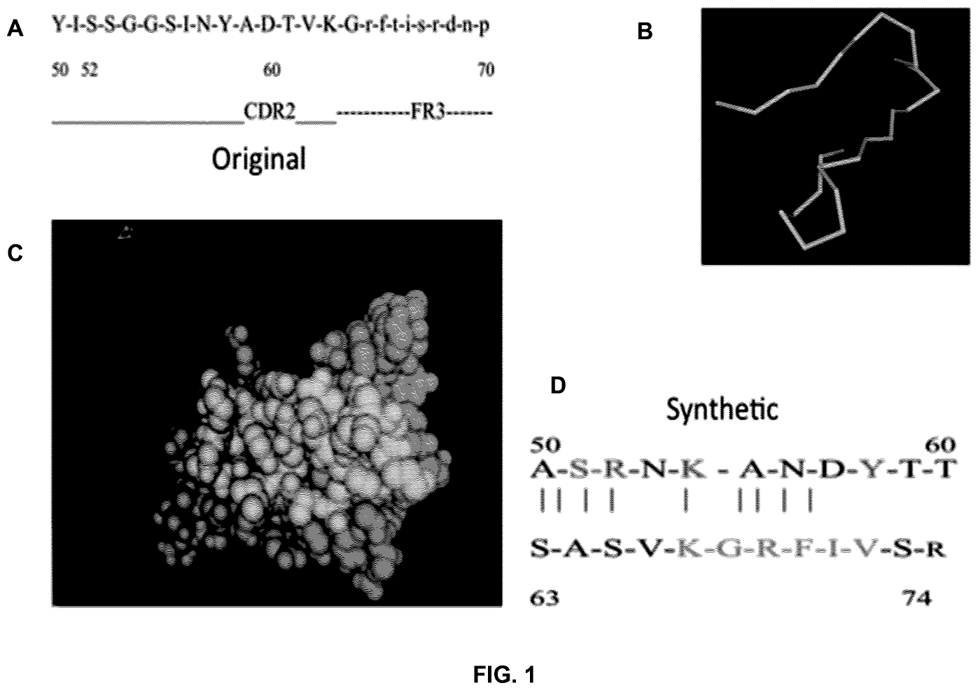

As shown in FIG. 1, the T15 peptide has a preference to form a hairpin structure at the C-terminus, which might impair its ability to self-bind (FIG. 1B). In the original HD antibody (T15), the HD sequence was inserted into the latter portion of the CDR3 and the first portion of CH1, a position that does not allow for secondary conformation. We hypothesized that in a recombinant form of an antibody with the HD peptide inserted at the c-terminus of the antibody, that the HD peptide was free to form the hairpin structure, reducing self-binding and its ability to potentiate therapeutic activity of antibodies through lattice formation. Similarly, the T15 peptide chemically conjugated to antibody, in the normal N to C-terminus configuration, would also have the ability to form a secondary conformation with reduced self-binding. In addition, the small HD peptide sequence, in the context of the large antibody protein, might have steric limitations to self-binding.

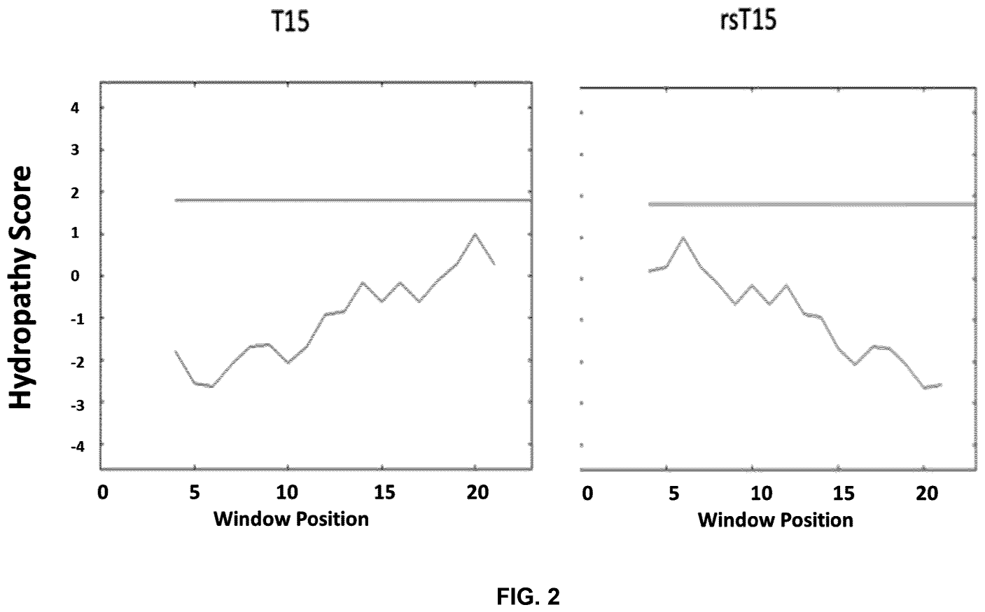

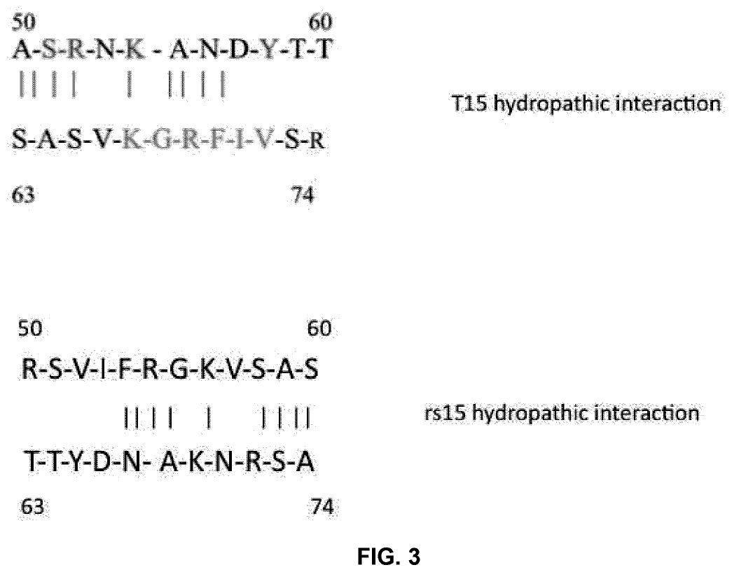

The first comparison was of the known T15 peptide configuration in normal or reverse order (see FIGS. 2 and 3). The examination of the original T15 sequence (left panel of FIG. 2) illustrates that self-binding correlates with the sequence hydropathy plot of the peptide. The first half of the peptide was derived from the CDR2 of the antibody while the second half was derived from the framework between CDRs. The first half then would contain the amino acids that confer self-binding and which are altered from the germline sequence; as important the more hydropathic residues of the second half serve to bind to the less hydropathic residues in the first half to initiate the self-binding process. Surprisingly when we examined the 24 mer peptide in reverse sequence (right panel of FIG. 2), it was also able to assume the required self-binding configuration despite the presence of the hairpin structure at the c-terminus. This then allows the tethering of the peptide through the former c-terminus (now n-terminus) to the antibody with the more important first half of the peptide, which determines self binding, tethered further from the antibody and more able to seek out the corresponding self-binding sequences in other peptide/antibody conjugates.

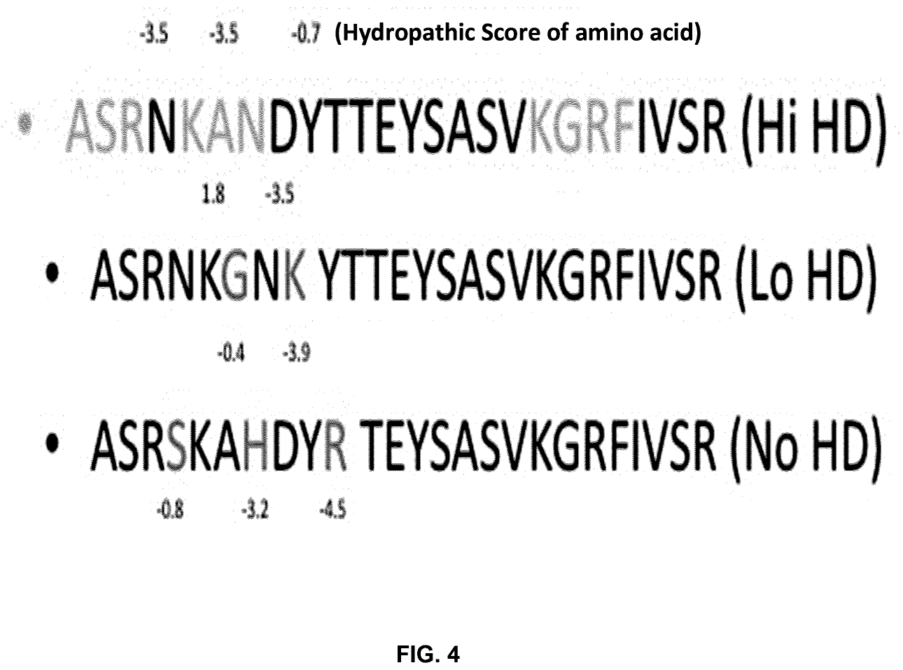

Identifying the hydropathic contribution of individual amino acids to self-binding in order to construct an optimizable motif, was done by comparing amino acid differences in 3 germline sequences drawn from the heavy chain CDR2/framework3 of Mopc antibodies with no, little or high HD activity. Individual amino acid hydropathic scores of amino acids (available at: http://gcat.davidson.edu/DGPB/kd/aminoacidscores.htm) were plotted to known positions of amino acids involved in self binding (see FIG. 4). As is shown, the amino acid changes in the T15 peptide (high HD activity) enabling self-binding are associated with distinctive changes in the hydropathic score of the substituted amino acid i.e. typically more hydrophilic substitutions enabling self binding to the more hydrophobic framework region. This allows one to predict that substitution of other amino acids into the former CDR2 region of the T15 peptide need to conserve the hydropathic character of the individual amino acids. It also became apparent that additional hydrophilic substitutions within the former CDR2 region of T15 peptide may increase the potential binding with the more hydrophobic former framework portion of the peptide and visa versa; substitution of more hydrophobic amino acids into the framework3 portion would allow more self binding to the more hydrophilic portion of the T15 peptide. This hydropathis-hydrophillic interaction serves to initiate the self-binding process which then results in the particular amino acids involved in self-binding, to form other non-covalent interactions. The whole process confers the relative affinity of the self-binding process. Some illustrative single amino acid, conservative substitutions are given in Table 2 (e.g. T15-var1, T15-var2 and T15-var3).

Without wishing to be bound by theory, it is believed that by increasing hydropathy of a HD peptide (for example by substitution of amino acids) the binding characteristics of the HD peptide may be modulated for example the binding characteristics of the HD peptide may be increased. It is further believed that the affinity for self-binding may be optimized to avoid the formation of antibody dimers before the binding of the antibody to a target. Therefore, the affinity of the HD peptide may be increased compared to a natural occurring HD peptide, but to still be less than the binding affinity of the antibody to its target. For example the biding affinity of the HD peptide may be 10⁻⁴, 10⁻⁵, 10⁻⁶, 10⁻⁷ or 10⁻⁸.

The potential increase in self-binding can be readily measured by synthetic peptide synthesis of single amino acid variants, labeling hydrogen atoms with tritium and then measuring binding to immobilized T15 peptide. Positive changes for singly amino acid substituted peptides then can be incorporated as double or triple amino acid changes. It must be pointed out that the final outcome is a self-binding peptide with higher self binding affinity but not so much that it causes aggregation of antibody.

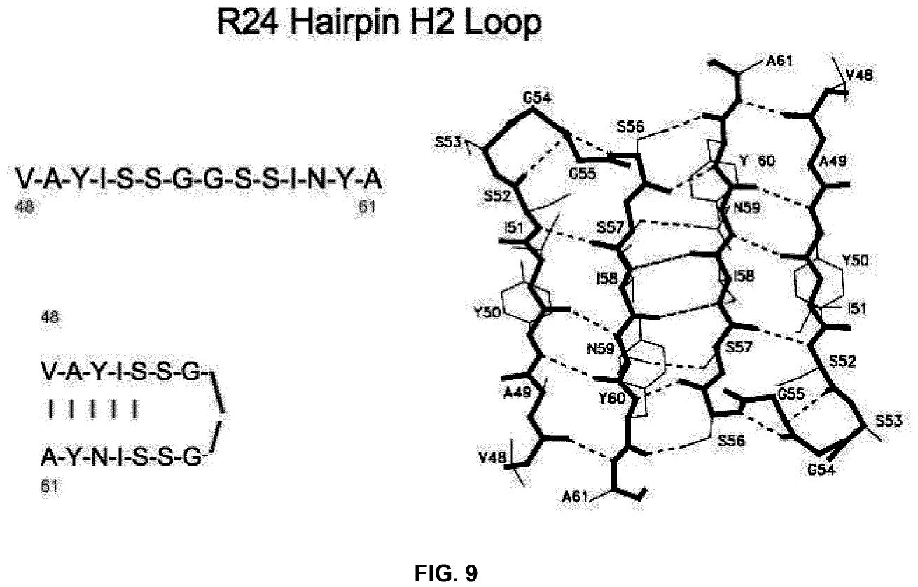

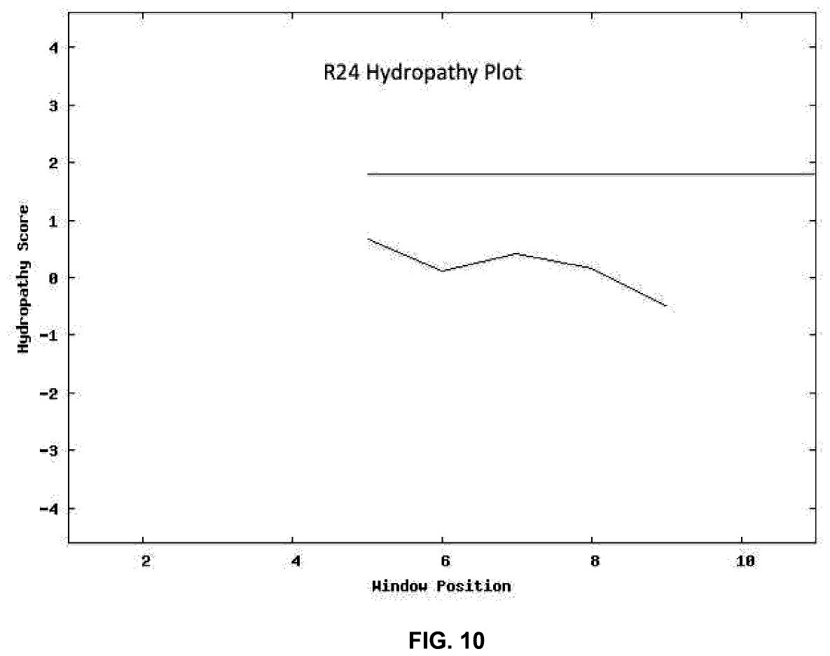

T15 is not the only naturally-occurring HD antibody and source of HD peptides. A well studied example is that of R24 directed to disialoganglioside GD3. As with T15, increased potency in the original murine antibody was associated with the presence of a self-binding peptide that allowed formation of lattices at the cell surface increasing therapeutic efficacy. As shown in FIG. 9, the R24 sequence, responsible for lattice formation will self-bind in anti-parallel fashion. As with T15, the R24 peptide though shorter than T15 demonstrates a corresponding hydropathic plot where one end of the peptide is more hydropathic than the other (see FIG. 10). The plot is basically a reversal of T15 where hydropathy goes from low to high, instead going from high to low. As such the same principles for promoting self-binding through reversal of the sequence, formation of dimers and making single, conservative amino acid substitutions with altered hydropathic character serve to increase self-binding.

### Example 2—Identification of Antibodies with Autophilic/Homologous Dimerization (HD) Activity

The following method uses the specific biophysical properties of antibodies incorporating an HD peptide to test for HD activity in an antibody preparation or in a phage library of scFv or Fab. The method can also be used as a quality assurance (QA) release assay for HD antibodies.

### Antibody Viscosity

Microdilution tubes (USA Scientific, Ocala, FL containing HPC G9 or HPC G11 at 1 mg/ml in PBS) were mounted on standard microscope slides. They were equilibrated at 4°, 20°, and 37° C., sealed and then positioned vertically and then horizontally then photographed at each temperature.

### Time Lapse Measurement of Viscosity Equilibrium

Microdilution tubes containing antibodies at 1 mg/ml in PBS were equilibrated at either 4 or 37° C. Tubes were positioned vertically for 3 s then horizontally and filmed. The time required for the meniscus to cease movement was measured using a stopwatch.

### Results

Temperature-dependent equilibrium of homodimers: HPC G9/HPC G11 are isoforms of IgG anti-phosphocholine antibodies whereby HPC G11 shares idiotype and sequence with CDR2/FR3 in TEPC-15. Accordingly, HPC G11 is homophilic and HPC G9 is not. Since temperature is known to affect the physical properties of proteins, including antibodies, the behavior of HPC G9 and HPC G11 was compared at 4, 20, and 37° C. using size-exclusion chromatography. The amount of antibody eluted within the exclusion volume was compared with that of the inclusion volume.

The ratio of excluded/included HPC G11 at different temperatures. There was no significant change in the ratio of excluded protein with HPC G9 at 4, 20, and 37° C. (data not shown), but the ratio for HPC G11 increased with temperature. These findings support the notion that the degree of dimerization of homophilic G11 in solution is higher at physiological temperature than at non-physiological temperatures.

### Viscosity Differences

The viscosity of HPC G9 and HPC G11 was also compared at different temperatures. A volume of 500 ml of each antibody was placed into racked tubes and allowed to equilibrate in a horizontal position at 4, 20, and 37° C. for 30 min. The tubes were then moved into a top-up, vertical position for 1 s and then returned to the horizontal position and photographed. The position of the meniscus was measured and the ratios of these measurements was calculated with HPC G9 or HPC G11. The ratio at 4° C. was 1.45, at 20° C. 0.92, and at 37° C. it was 0.54. The tilting of the tubes after equilibration was reversed (i.e. the bottom of the tube was raised vertically for 1 s and then returned to a horizontal position and photographed). The ratio of HPC G9 to HPC G11 at 4° C. was 1.5, 1.41 at 20° C., and 2.06 at 37° C.

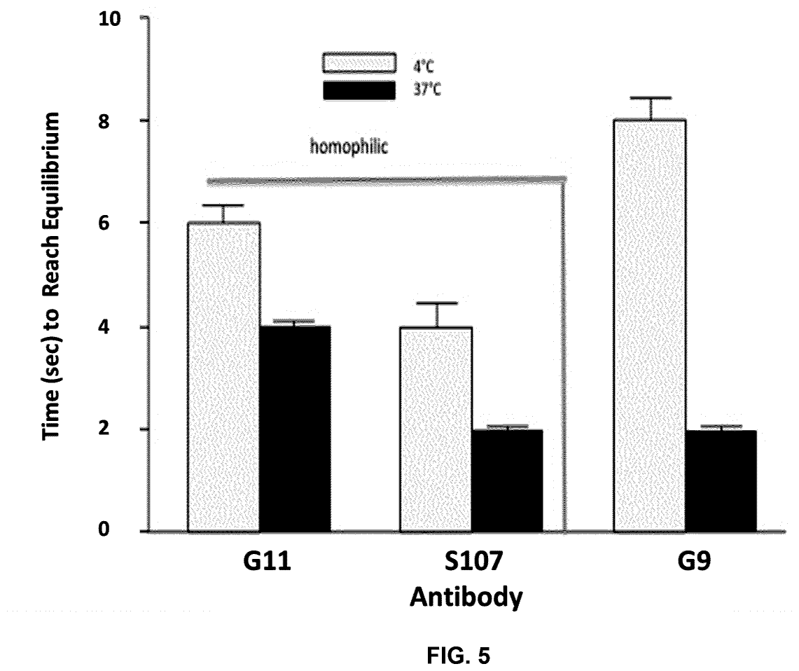

To exclude the possibility that the viscosity of Gll is unique, S107 a murine monoclonal IgA antibody known to be hemophilic was tested. For comparison, another murine antibody G9 was included in the viscosity analysis. The tubes were positioned first bottom-up for 3 s and then returned to a horizontal position. The time required for the meniscus to cease movement was measured for each antibody. In FIG. 5, we show the seconds recorded for the meniscus of these antibodies to cease movement at 4 and 37° C. The more time required for movement to cease, the higher the viscosity of the solution. The time differences recorded at both temperatures as shown in FIG. 5 are smaller for G11 and S 107 than for G9. The ratio of time at 4° C. divided by time at 37° C. for G11, S 107, and 1F7 are identical, while the ratio for G9 is more than 2 times greater. G11 and S107 are facultative homophilic polymers, while 1F7 IgM is a covalent pentamer. It is interesting to note that the homophilic polymers and the covalent IgM polymer have similar viscosity but different viscosity from the monomeric G9 bivalent antibody. This unique viscosity of homophilic antibodies is independent of Ig class and might be part of their unique dimerization potential responsible for their superior targeting.

### Example 3—Production and Characterization of a Recombinant Therapeutic HD Antibody (Rutuximab) with Reverse Configuration

The following example illustrates production of a reverse sequence HD antibody and the changes in therapeutic characteristics of a recombinant HD form of an antibody compared to the native antibody. To test the capacity of the HD peptide, molecular biological techniques were used to generate a chimeric version of the Rituximab antibody (chRituximab), and a chimeric HD antibody that is identical to chRituximab, except for the addition of the HD peptide (rs-T15) to the C-terminus of each variable region of the heavy chain.

### Materials and Methods

### Cell lines

JOK-1 cells were a gift of Affimed Inc. JOK-1 cells were grown in RPMI-1640 with Glutamax (Gibco), supplemented with 10% FBS-Premium-HI (Aleken Biologicals), and 1% Penicillin/Streptomycin (Gibco). Raji, and Ramos, cells were obtained from the American Type Culture Collection (ATCC), numbers HB-9645, CCL-86, CRL-1596, and TIB-152, respectively. Raji and Ramos cells were maintained in RPMI-1640 Medium with HEPES (ATCC), supplemented with 10% FBS-Premium-HI (Aleken Biologicals), and 1% Penicillin/Streptomycin (Gibco). Rituximab cells were maintained in RPMI-1640 Medium with HEPES (ATCC), supplemented with 10% FBS-low-IgG (Gibco), 1% Penicillin/Streptomycin (Gibco), and 0.5% Glutamax (Gibco). CHO—S cells were purchased from Invitrogen, and were grown in CD CHO medium, supplemented with 1% HT supplement (Gibco), 2% Glutamax (Gibco), and 100 U/ml pen/strep (Gibco). After introduction of vector DNA, CHO—S cells were grown as above with the addition of 1.2 mg/ml 418 (Invivogen) for selection. All cells were maintained at 37oC and 5% CO2.

### Construction of Chimeric Antibody Genes

Heavy and Light chain variable regions were synthesized from published Rituximab sequences:

Rituximab heavy chain:
(SEQ ID NO: 20)
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKAEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K
Rituximab light chain:
(SEQ ID NO: 21)
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

The Rituximab heavy chain variable regions were amplified from the cDNA pool by PCR using primers modVHRituximabfwd and modVHRituximabrev. All oligos were purchased from Operon. The Rituximab light chain variable regions were amplified from the cDNA pool by PCR using primers modVLRituximabfwd and modVLRituximabrev. The heavy chain and light chain PCR products were cloned into the XhoI-NheI and SacI-HindIII sites, respectively, of vector pAc-k-CH3 (Progen Biotechnik GmbH), to form pAc-k-RituximabH and RituximabK. Clones were verified by sequencing in both directions. All restriction enzymes were purchased from Takara or New England Biolabs. Taq polymerase (Promega) was used for all PCR. All enzymatic reactions were carried out using manufacturers' protocols.

### Construction of Antibody Expression Vectors

Oligos LongT15fwd, LongT15rev, and PrimerB were used in a nested PCR to construct a DNA sequence that encodes the reverse sequence of T15 peptide. The resulting PCR product was cloned into the SalI-NotI sites in MCSB of pIRES (Clontech) to form pHD. The complete heavy and light chains of pAc-k-RituximabH and pAc-k-RituximabK were PCR amplified using primers modVHXfwd and modVHXrev, or VKXfwd and VKXrev, respectively. The light chain was cloned into the Nhel-Xhol sites of MCSA of vector pHD, and the heavy chain was cloned into the SalI-NotI sites of the resulting vector to form pchRituximab-HD. Clones were verified by sequencing in both directions. To produce pchRituximab (anti-CD20 without the T15 peptide), pchRituximab-HD and pIRES were digested with Notl and Clal. Resulting DNA fragments of ˜6 Kb from pchRituximab-HD, and ˜2.2 Kb from pIRES were each gel purified from a 1% agarose gel using a Qiaquick kit (Qiagen), and ligated together to form Rituximab. Clones were verified by sequencing in both directions. All vector constructs were introduced into E. coli (XL-10 cells, from Stratagene) using the provided heat shock protocols. Plasmids were purified from 3 ml of overnight bacterial culture using a Qiagen mini-prep kit. Vectors pchRituximab and pchRituximab-HD were electroporated into CHO—S cells using a 4 mm gap cuvette in an Eppendorf Multiporator set to 580 V and 40 μs. Two days of recovery were allowed before the start of selection.

### Purification of Recombinant Antibodies

Cell culture supernatant was harvested every 3-5 days, depending on cell density. Cell suspensions were centrifuged at low speed (480-740×g) for 7 to 10 minutes, and the supernatant was held at −20° C. prior to additional processing. After rapid thawing at 37° C., supernatant was passed through a 0.2 μm filter (Corning) by vacuum filtration to remove cell debris, and filtered supernatant was then passed over HiTrap Protein G HP column (GE Healthcare). Bound antibodies were eluted with 0.1 M glycine buffer pH 2.7, collected in ImL fractions, and the pH was neutralized with 50 μL 1M Tris pH 9. Elution profile was determined by reading UV absorbance at 280 (data not shown). Fractions with significant protein content were then pooled and concentrated using Amicon Ultra centrifugal filtration device 50,000 MW cutoff (Millipore) according to the manufacture's instructions.

### Cell Surface Binding

3×10⁵ per well of Raji, Ramos, or JOK-1 cells were seeded in a 24 well plate and incubated overnight at 37° C. and 5% CO₂. Cells were then harvested and washed twice with PBS. Cells were resuspended in ImL PBS and were incubated with either chRituximab or HD chRituximab at increasing concentrations (1 μg, 5 μg, 10 μg, 20 μg/mL) and incubated at 4° C. for 30 minutes. Excess antibody was removed by washing cells twice with PBS, and then cells were resuspended in a 1 mL solution of FITC conjugated goat anti-Human (Sigma, 1:1000) and incubated at 4° C. for 30 minutes. After washing twice, cells were resuspended in 200 μL PBS and analyzed by flow cytometry (BD FACSCalibur Instrument, BD Bioscience). Specific mean fluorescence intensity was determined by using the formula: specific MFI=MFI (primary Ab+goat anti-Human FITC)−MFI (goat anti-Human FITC).

### Apoptosis Assay

2×10⁵ per well of Raji, Ramos, or JOK-1, cells were seeded in a 24 well plate and incubated overnight at 37° C. and 5% CO₂. Cells were then treated with increasing concentrations of Abs for 20 hours at 37° C. Cells were harvested, washed once with PBS, and resuspended with 100 μL 1 X annexin binding buffer containing 3 μL annexin V Alexa Fluor 488 conjugate (Invitrogen) and propidium iodide (Sigma) at a final concentration of 4 μg/mL to detect apoptosis and cell death, respectively. After 20 minutes incubation at 37° C., cells were diluted with 150 μL of 1 X annexin binding buffer and analyzed by flow cytometry (BD FACSCalibur Instrument, BD Bioscience). Percent apoptotic cells was determined by gating the healthy population in the untreated control samples and using the formula: Percent Apoptotic Cells=(1−(Live Treated Target Cells/Live Untreated Target Cells))*100.

### CDC Assay

2×10⁵ cells were seeded into a 24 well plate and incubated overnight at 37° C. and 5% CO₂. Cells were then treated with increasing concentrations of Abs for 2 hours at 37° C. in the presence of 5% rabbit HLA-ABC complement enriched sera (Sigma). Cells were harvested and washed once with PBS, resuspended in 200 μL of PBS containing 50 nM calcein-AM (Biochemica) and 4 μg/mL propidium iodide (Sigma). After incubation for 20 minutes at 37° C. cell viability was analyzed by flow cytometry (BD FACSCalibur Instrument, BD Bioscience). Percent killing was determined by the formula: Percent Dead Cells=(1−(Live Treated Target Cells/Live Untreated Target Cells))*100.

### PBMC Separation

Peripheral blood mononuclear cells (PBMC) were prepared from healthy donors' buffy coat (Kentucky Blood Center, Lexington KY) by Ficoll-Hypaque density gradient centrifugation. PBMC were diluted to 6×10⁶ cells/mL in hRPMI (10% FBS, low IgG) culture media and maintained for a maximum of three days. PBMC viability and day-to-day cell population variation was analyzed by flow cytometry (BD FACSCalibur Instrument, BD Bioscience) before experimentation.

### ADCC Assay

Target cells (Raji, Ramos, or JOK-1) were harvested from T75 flasks and resuspended in 1 mL of media containing 400 nM calcein-AM (Biochemica) and 8 μL of TFL2 dye (OncoImmunin), used according to manufacturer's instructions. Target cells were labeled for 45 minutes at 37° C., washed twice in media, and resuspended to a density of 6×10⁵ cells/mL. Effector cells (PBMC) were then harvested from T75 flasks and resuspended to a density of 1.2×10⁷ cells/mL. Target cells and effector cells were mixed at an E:T ratio of 20:1 then 250 μL of cell mixture was aliquoted into individual 5 mL round bottom tubes and incubated with increasing concentrations of Abs for 2 hours at 37° C. After incubation, target cell viability was analyzed by flow cytometry (BD FACSCalibur Instrument, BD Bioscience). Percent killing was determined by the formula: Percent Dead Cells=(1−(Live Treated Target Cells/Live Untreated Target Cells))*100.

### Results

### Florescence Activated Cell Sorting (FACS)

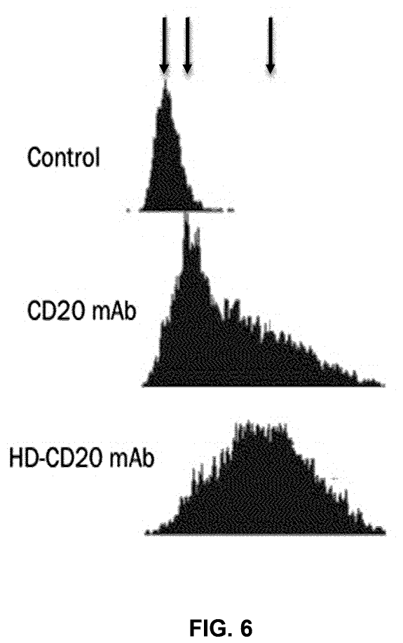

To verify that the recombinant chRituximab and chRituximab-HD antibodies are functional, their ability to bind to cells from the human B-cell JOK-1 line was tested, using fluorescence activated cell sorting (FACS). In FIG. 6, the lower panel shows the mean fluorescence intensity (MFI) of staining with the chRituximab-HD antibody, while the upper panel represents the staining using the chRituximab, non-HD antibody. Binding of the chRituximab-HD antibody was approximately four-fold higher than binding of chRituximab.

### Induction of Apoptosis is Dependent on Receptor Cross-Linking

One of the proposed mechanisms of HD antibodies is receptor crosslinked induction of apoptosis. The induction of apoptosis of the chRituximab and HD antibodies were compared in three cell lines Raji, Ramos and JOK-1. The addition of chRituximab induces apoptosis in approximately 30% of the cells in some cell lines especially at the highest doses. The HD antibody induces significantly more apoptosis than the unmodified chimeric. Similarly, the HD antibody is a more potent inducer of apoptosis in Ramos cells and other B lymphoma cells. Table 4 shows the apoptotic effects of the two versions of Rituximab over a range of concentrations. It is interesting to note, at lower concentration of Abs the enhancing effect is much more pronounced. For example, after treatment of Raji cells with 5 μg/mL of either antibody, the percent of apoptotic cells is 2.5 fold higher after HD treatment, but it is slightly less than 2-fold higher after treatment with 20 μg/mL.

TABLE 4
Comparison of Induction of Apoptosis

| Cell Line | Antibody dose¹ | chRituximab² | | HD-ch-Rituximab | |
|---|---|---|---|---|---|
| Raji | 1 | 0.83 | (2.18) | 5.06 | (2.16) |
| | 5 | 14.90 | (1.81) | 36.91 | (8.73) |
| | 10 | 26.73 | (4.28) | 47.40 | (2.89) |
| | 20 | 30.05 | (3.13) | 58.37 | (4.67) |
| Ramos | 1 | 4.00 | (0.11) | 19.36 | (2.06) |
| | 5 | 20.11 | (2.30) | 33.06 | (7.1) |
| | 10 | 24.61 | (0.40) | 42.53 | (4.28) |
| | 20 | 31.74 | (1.70) | 40.79 | (1.41) |
| Jok-1 | 1 | 7.85 | (0.99) | 4.39 | (0.99) |
| | 5 | 23.77 | (5.48) | 27.19 | (12.14) |
| | 10 | 59.43 | (13.89) | 52.13 | (18.97) |
| | 29 | 49.44 | (7.50) | 56.87 | (4.60) |

¹Differing amounts of antibodies were added for 20 hours to each cell line (μg/ml)
²Percent apoptotic cells induced by chRituximab (Number in parenthesis = plus/minus)
³Percent apoptotic cells induced by HD-chRituximab (Number in parenthesis = plus/minus

### Comparison of Complement Dependent Cytotoxicity (CDC)

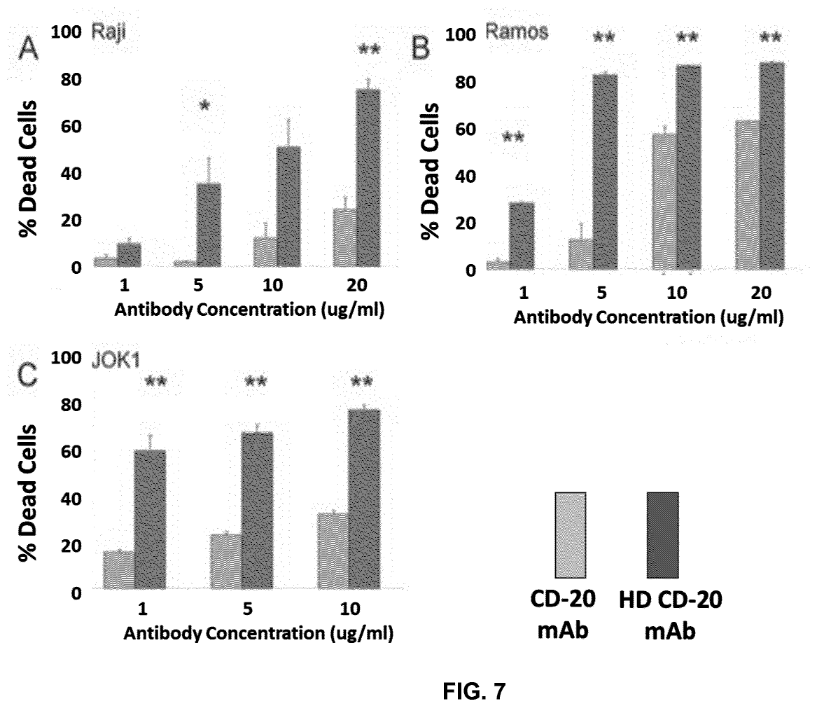

The CDC activity of the chRituximab and chRituximab-HD was compared (see FIGS. 7A-C). CDC is induced after binding of complement components to the Fc region of an antibody, and is potent in the IgG1 isotype, which is the isotype of the HD construct. An enhancing effect was observed in all cell lines. As seen in FIG. 7A, for example, at 5 μg/mL there was virtually no CDC activity in Raji cells with the chimeric, however, 35% of cells were killed with the HD mAb. This correlates to the highest improvement of effectiveness in apoptosis. It is interesting to note that the potency of the HD antibody plateaus at 5 μg/ml in Ramos cells (FIG. 7B). The chRituximab appears to plateau at 10 μg/ml, but it does not reach the potency of the HD Ab at any level tested, suggesting that even higher doses would not reach the killing capacity of 5 μg/ml HD Ab.

### Comparison of ADCC

The chimeric antibody was tested in its ability to induce antibody-dependent cellular cytotoxicity (ADCC). The HD antibody induces significantly more ADCC than chRituximab in Raji and Ramos cells at 1 μg/ml and 3 μg/ml.

### Inhibition of Lymphoma Growth In Vitro

To approximate the in vivo killing potential of these anti-CD20 antibodies on tumor cells, the anti-proliferative effects of the chRituximab and chRituximab-HD was tested in Raji and Ramos cell lines. The assay measures the level of fluorescence dye binding to nucleic acid (see Methods and Materials). The HD antibody inhibited proliferation to a greater extent in both cell lines at all concentrations tested.

### Example 4—In Vivo Characterization of a HD Anti-Her-2 Antibody (Trastuzumab) with Reverse Configuration

An HD form of Herceptin (Trastuzumab) was produced as described in Example 3. The CDR sequences were drawn from the heavy and light chain variable regions:

Herceptin Light chain (1 and 2)
(SEQ ID NO: 22)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Herceptin Heavy chain (1 and 2)
(SEQ ID NO: 23)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K

To evaluate HD-antibodies for efficacy in a relevant human tumor model, therapy was initiated in a nude mouse model of a low antigen expressing breast cancer (MCF-7), 7 days after injection of tumor

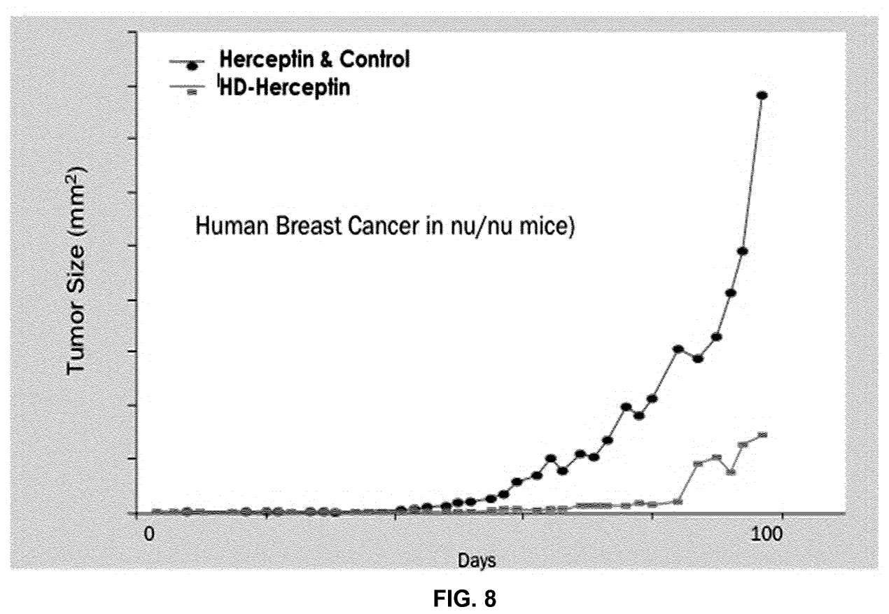

As shown in FIG. 8, Herceptin showed no therapeutic effect, with tumor measurements the same as the control (top line). In contrast, HD-Herceptin suppressed tumor growth dramatically (bottom line). The lack of activity of Herceptin in this model was expected as it does not recognize low antigen expressing breast cancer. Upon histological examination of tumors treated with HD-Herceptin, few viable cancer cells were found while inflammatory cells were in abundance.

When extrapolated to human doses, it was found that the maximal active dose of HD-Herceptin was 10 μg while Herceptin had no effect up to 100 μg/dose (based on the assumption of a 75 kg human subject).

### Example 5—Characterization of a HD Anti-Vegf (Avastin) Antibody with Reverse Configuration

A biosimilar Bevacizumab antibody was converted into its recombinant HD form as described in Example 3 utilizing CDRs drawn from the variable heavy and light chain sequences:

Bevacizumab light chain
(SEQ ID NO: 24)
DIQMTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Bevacizumab heavy chain
(SEQ ID NO: 25)
EVQLVESGGGLVQPGGSLRLSCAASGYTFTNYGMNWVRQAPGKGLEWVGW
INTYTGEPTYAADFKRRFTFSLDTSKSTAYLQMNSLRAEDTAVYYCAKYP
HYYGSSHWYFDVWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK

VEGF is produced by healthy and neoplastic cells. Its activities are mediated by two receptor tyrosine kinases. VEGF signaling is often a rate-limiting step in physiologic and pathologic angiogenesis. Bevacizumab has been studied as an antiangiogenic cancer therapeutic as a single agent and in combination with chemotherapy in patients with stage III and IV colon cancer.

Summation of in vivo tumor model evaluation of HD Bevacizumab:
a) Inhibits tumor growth (in renal cell carcinoma and in prostate cancer);
b) Regresses tumors (in colon carcinoma);
c) Prevents tumor recurrence);
d) Reduces/prevents metastases (in both renal and colon metastases);
e) Reduces growth of A498 human renal carcinoma cells.

The in vivo evaluation indicates that other types of cancer can be treated with the HD ant-Vegf and that it not only inhibits proliferation but regresses cancers.

### Example 6—Characterization of an HD anti-PD-L1 Antibody with T15 Dimer Configuration

CDRs from the variable heavy and light chain sequences below of Atezolizumab (anti-PD-L1 mAb) was used to construct a humanized IgG1 (as described in Example 3) incorporating the reverse configuration of the T15 dimer (see Table 2) onto the Fc end of the antibody.

Heavy chain:
(SEQ ID NO: 26)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
Light Chain:
(SEQ ID NO: 27)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

HD-Atezolizumab was tested versus the parental antibody against melanoma cells bearing the PD-L1 antigen. Expression of human PD-L1 was assessed by flow cytometry. In brief, melanoma cell suspensions were prepared and washed in fluorescence-activated cell sorter buffer consisting of phosphate-buffered saline (PBS; pH 7.2) containing 2% fetal bovine serum. Cells were incubated with anti-PD-L1 antibody (2 μg/mL), or mouse IgG1 isotype control (2 μg/mL) for 60 minutes at 4° C., washed three times, and incubated with FITC-labeled secondary antibody (BD PharMingen, San Jose, CA) for 30 minutes at 4° C. The cells were then washed three additional times in PBS, fixed in 2% formalin, and assessed for fluorescence (FACScalibur flow cytometer; BD Bioscience, San Diego, CA).

Results were expressed as the Mean Fluorescence Index (MFI) or Fold increase in Binding (with background subtracted)—see Table 5 below.

TABLE 5

| FITC (background) | Atezolizumab | HD-Atezolizumab | Fold Increase |
|---|---|---|---|
| 5 | 27 | 2498 | 113 |

The results demonstrate over a 100 fold increase in binding to cell surface PD-L1 with the HD form of the antibody. This will necessarily result in increased ability to block tumor cell mediated T-cell suppression.

### Example 7—HD Technology for In Vitro Diagnostic Applications

A monoclonal antibody to PSA was HD-modified by recombinant technology and then labeled for electro-chemilumnescence detection and utilized in a antigen detection assay (Anal. Sci. 2009 May 25 (5): 587-97). The non-modified antibody was compared to the HD-modified one, wherein the T15 peptide was linked to the end of the light chain of the antibody.

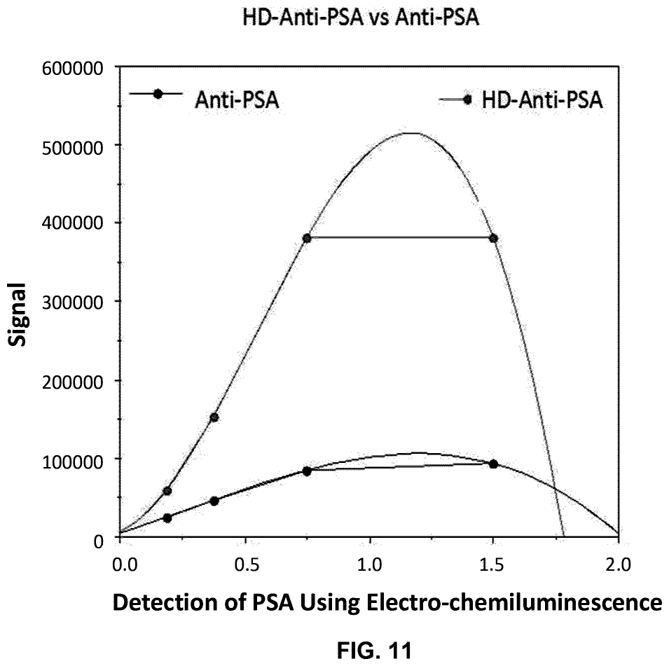

The HD-modified antibody provided a much higher signal to noise ratio than the non-modified antibody making the assay more sensitive especially at lower antigen concentrations (see FIG. 11).

The results demonstrate that HD-modified antibodies can be used in any configuration (direct or indirect detection), and with any method of detection including the more standard ELISAs. HD-modified antibodies can be used to enhance signal as well as reduce the time to read-out of assays due to the higher signal generation.

The use of the word “a” or “an” when used herein in conjunction with the term “comprising” may mean “one,” but it is also consistent with the meaning of “one or more,” “at least one” and “one or more than one.” Any element expressed in the singular form also encompasses its plural form. Any element expressed in the plural form also encompasses its singular form. The term “plurality” as used herein means more than one, for example, two or more, three or more, four or more, and the like.

As used herein, the terms “comprising,” “having,” “including” and “containing,” and grammatical variations thereof, are inclusive or open-ended and do not exclude additional, un-recited elements and/or method steps. The term “consisting essentially of” when used herein in connection with a composition, use or method, denotes that additional elements, method steps or both additional elements and method steps may be present, but that these additions do not materially affect the manner in which the recited composition, method or use functions. The term “consisting of” when used herein in connection with a composition, use or method, excludes the presence of additional elements and/or method steps. As used herein, the term “about”, when used to describe a recited value, means within 10% of the recited value.

All citations are hereby incorporated by reference.

The present invention has been described with regard to one or more embodiments. However, it will be apparent to persons skilled in the art that a number of variations and modifications can be made without departing from the scope of the invention as defined in the claims. The scope of the claims should not be limited by the preferred embodiments set forth in the examples, but should be given the broadest interpretation consistent with the description as a whole

What is claimed is:

1. A homologous dimerization (HD) peptide comprising an amino acid sequence in reverse configuration compared to a corresponding naturally occurring HD peptide, or conservative variants thereof.

2. The HD peptide of claim 1, wherein the corresponding naturally occurring HD peptide is a T15 HD peptide or a R24 HD peptide.

3. The HD peptide of claim 1 or claim 2, wherein the HD peptide comprises an amino acid sequence having at least 90% sequence identity to SEQ ID No. 4, SEQ ID No. 6 or SEQ ID NO: 18.

4. The HD peptide of any one of claim 1-3, having one or more than one amino acid substitutions wherein the one or more than one substitution increases the hydropathy of the HD peptide.

5. A homologous dimerization (HD) peptide comprising one or more than one amino acid substitutions wherein the one or more than one substitution increases the hydropathy of the HD peptide.

6. The HD peptide of claim 5, wherein the HD peptide comprises the amino acid sequence of SEQ ID No: 1, having one or more than one amino acid substitutions at positions 4, 7, and 18.

7. The HD peptide of claim 6, wherein the one or more than one amino acid substitution at position 4 is to K or a conserved substitution of K, the one or more than one amino acid substitutions at position 7 is to R or a conserved substitution of R and the one or more than one amino acid substitutions at position 18 is to H or a conserved substitution of H.

8. The HD peptide of claim 6 or 7, wherein the HD peptide comprises an amino acid sequence having 80%-100% sequence identity to SEQ ID NO: 8, 9 or 10.

9. A homologous dimerization (HD) peptide dimer comprising a first HD peptide and a second HD peptide, wherein the first and second HD peptide are derived from a naturally occurring HD peptide or wherein the first and second HD peptide are derived from a reverse sequences of a naturally occurring HD peptide.

10. The homologous dimerization (HD) peptide dimer of claim 9, wherein the first and/or second HD comprises the HD peptide of any one of claims 1-8.

11. The HD peptide dimer of any one of claim 9 or 10, wherein the first and second HD peptides of the dimer are joined by a linker.

12. The HD peptide dimer of claim 11, wherein the linker is gly-gly.

13. The homologous dimerization (HD) peptide dimer of claim 9, wherein the HD peptide dimer comprises an amino acid sequence having 80-100% sequence identity to SEQ ID NO: 5, 7, 11, 12, 13, 14, 15, 17 or 19.

14. An antibody or antigen-binding fragment comprising the HD peptide of any one of claims 1 to 8 or the HD peptide dimer of any one of claims 9-13.

15. The antibody or antigen-binding fragment of claim 14, wherein the antibody or antigen-binding fragment is a humanized IgG.

16. The antibody or antigen-binding fragment of claim 16, wherein the antibody or antigen-binding fragment is a humanized IgG1, humanized IgG4 or humanized IgG3.

17. The antibody or antigen-binding fragment of any one of claims 14 to 16, wherein the HD peptide or HD peptide dimer is fused to a nucleotide affinity site of the antibody or antigen-binding fragment.

18. The antibody or antigen-binding fragment of claim 17, wherein the HD peptide or HD peptide dimer is fused through lysines, cysteines or carbohydrates.

19. The antibody or antigen-binding fragment of any one of claims 14 to 18, wherein the HD peptide or HD peptide dimer is positioned immediately following the CDR3 of the heavy chain or light chain of the antibody.

20. The antibody or antigen-binding fragment of any one of claims 14 to 18, wherein the HD peptide or HD peptide dimer is positioned immediately following the C-terminal of a heavy chain or light chain constant region of the antibody.

21. The antibody or antigen-binding fragment of any one of claims 14 to 18, wherein the HD peptide or HD peptide dimer is positioned immediately following the heavy chain variable region of the antibody.

22. The antibody or antigen-binding fragment of any one of claims 14 to 18, wherein the HD peptide or HD peptide dimer is positioned immediately following the C-terminal of a Fc region of the antibody.

23. The antibody or antigen-binding fragment of any one of claims 14 to 22, wherein the HD peptide or HD peptide dimer is conjugated to the antibody.

24. The antibody or antigen-binding fragment of any one of claims 14 to 23, wherein the antibody is a chimeric recombinant antibody.

25. The antibody or antigen-binding fragment of any one of claims 14 to 24, wherein the HD peptide or HD peptide dimer is preceded by a spacer.

26. The antibody or antigen-binding fragment of claim 25, wherein the spacer is gly-gly.

27. The antibody or antigen-binding fragment of any one of claims 14 to 26, wherein the antibody is a single-chain antibody (scFvs), bi-specific antibody (BsAbs) or antibody-like peptide.

28. The antibody or antigen-binding fragment of any one of claims 14 to 27, wherein the antibody is a humanized monoclonal antibody.

29. The antibody or antigen-binding fragment of any one of claims 14 to 28, wherein the antibody is a Her-2neu antibody (such as Herceptin), a CD-20 antibody (such as Rituxin), a vascular endothelial growth factor antibody (such as Avastin) or is a checkpoint inhibitor antibody (such as PD-L1).

30. A composition comprising one or more antibody or antigen-binding fragment according to any one of claims 14 to 29, and a pharmaceutically acceptable carrier.

31. An expression vector comprising a first nucleic acid sequence encoding the HD peptide of any one of claims 1 to 8 or the HD peptide dimer of any one of claims 9-13.

32. The expression vector of claim 31, further comprising a second nucleic acid sequence encoding an antibody or antigen binding fragment, such that when the first and second nucleic acid sequences are expressed the HD peptide and the antibody or antigen binding fragment are expressed as a fusion protein.

33. A method of generating an homologous dimerization (HD) antibody, comprising expressing the expression vector of claim 32 in a host cell.

34. An isolated host cell transformed with the expression vector of claim 31 or 32.

35. A method of enhancing binding and/or potency of an antibody, comprising:
conjugating the HD peptide of any one of claims 1 to 8 or the HD dimer of any one of claims 9-13 to the antibody: or
recombinantly expressing a chimeric antibody comprising the HD peptide.

36. A method of treating a patient suffering from a disease or condition, comprising administering an antibody or antigen binding fragment according to any one of claims 14 to 29.

37. The method of claim 36, wherein the disease or condition is one selected from the list consisting of cancer, auto-immune disorders, inflammatory disorders, neurodegenerative disease, cardiovascular disease, graft or transplant rejection.

38. A method of detecting an analyte in a sample, comprising:
contacting the analyte with an antibody or antigen binding fragment directed to the analyte, wherein the antibody or antigen binding fragment is fused to the HD peptide of any one of claims 1 to 8 or the HD peptide dimer of any one of claims 9 to 13; and
detecting a complex formed by the analyte and the antibody fused to the HD peptide.

39. A kit for detecting an analyte in a sample, comprising:
an antibody or antigen binding fragment directed to the analyte, the antibody or antigen binding fragment fused to the HD peptide of any one of claims 1 to 8 or the HD peptide dimer of any one of claims 9-13; and
instructions for use in detecting the analyte.

40. A phage display library comprising an antibody or antigen binding fragment linked to a HD peptide according to any one of claims 1 to 8, or the HD peptide dimer of any one of claims 9 to 13.

41. Use of the antibody according to any one of claims 14 to 29 in therapy.

42. The antibody according to any one of claims 14 to 29 for use in therapy.

43. Use of the antibody of any one of claims 14 to 29 in a diagnostic test.

44. The antibody according to any one of claims 14 to 29 for use in a diagnostic test.
